(19) United States
(12) Patent Application Publication
(10) Pub. No.: US 2010/0022509 A1
(43) Pub. Date: Jan. 28, 2010

(54) Inhibitors of MshC and Homologs Thereof, and Methods of Identifying Same

(76) Inventors: Robert C. Fahey, Del Mar, CA (US); Gerald L. Newton, San Diego, CA (US); Philong V. Ta, Walnut, CA (US); Nancy Buchmeier, Encinitas, CA (US)

Correspondence Address: DLA PIPER LLP (US), 4365 EXECUTIVE DRIVE, SUITE 1100, SAN DIEGO, CA, 92121-2133, US

(21) Appl. No.: 12/300,236

(22) PCT Filed: Jun. 7, 2007
(86) PCT No.: PCT/US07/13558

§ 371 Date: Oct. 6, 2009

Related U.S. Application Data
(60) Provisional application No. 60/811,994, filed on Jun. 7, 2006.

(51) Int. Cl. A61K 31/554; C07D 281/12; C12Q 1/02; A61P 31/04

(52) U.S. Cl. 514/211.04; 540/488; 435/29

(57) ABSTRACT

The present invention utilizes three families of bacterial enzymes, which play a key role in mycothiol biosynthesis. The three families are bacterial cysteine:glucosaminyl inositol ligases (MshC) with catalytic ligase activity for ligation of glucosaminyl inositol and cysteine, bacterial acetyl-CoA:Cys-GlcN-Ins acetyltransferases (MshD) with catalytic activity for addition of an acetyl group to Cys-GlcN-Ins and bacterial MshA glycosyltransferase with catalytic activity for production of GlcNAc-Ins. The invention provides methods for using the mycothiol biosynthesis ligases, acetyltransferases or glycosyltransferases in drug screening assays to determine compounds that inhibit activity. The invention also provides inhibitors of the production or activity of the enzymes of mycothiol biosynthesis, and use of the inhibitors for treating microbial infection.

### BACKGROUND OF THE INVENTION

1. Field of the Invention

The invention relates generally to identification of inhibitors of three families of enzymatic compounds produced by bacteria and involved in the steps of mycothiol biosynthesis and, more specifically, to identification of inhibitors of MshC, MshD and MshA and methods of use thereof, especially for use in drug discovery and disease control.

2. Background Information

Glutathione (GSH) is the dominant low molecular weight thiol in most eukaryotes and Gram-negative bacteria, and it plays a key role in protection of the cell against oxygen toxicity and electrophilic toxins. However, most gram-positive bacteria, including many strict aerobes, do not produce glutathione. Yet aerobic organisms are subjected to oxidative stress from many sources, including atmospheric oxygen, basal metabolic activities, and, in the case of pathogenic microorganisms, toxic oxidants from the host phagocytic response intended to destroy the bacterial invader.

Actinomycetes, including Streptomyces and Mycobacterium, do not make GSH but produce instead millimolar levels of mycothiol (MSH, AcCys-GlcN-Ins), an unusual conjugate of N-acetylcysteine (AcCys) with 1D-myo-inosityl 2-amino-2-deoxy-I-D-glucopyranoside (GlcN-Ins). The biochemistry of mycothiol appears to have evolved completely independently of that of glutathione. However, it has already been established that the metabolism of mycothiol parallels that of glutathione metabolism in several enzymatic processes. First, formaldehyde is detoxified in glutathione-producing organisms by NAD/glutathione-dependent formaldehyde dehydrogenase. An analogous process involving NAD/mycothiol-dependent formaldehyde dehydrogenase has been identified in the actinomycete Amycolatopsis methanolica, and the enzyme has been sequenced.

The second enzymatic process involves a mycothiol homolog of glutathione reductase recently cloned from M. tuberculosis and expressed in M. smegmatis. The reductase is reasonably specific for the disulfide of mycothiol, but is also active with the disulfide of AcCys-GlcN, the desmyo-inositol derivative of mycothiol. A general mycothiol-dependent detoxification process has been described in M. smegmatis in which MSH forms S-conjugates (MSR) with reactive electrophiles, including some antibiotics, and MSR is subsequently degraded by the enzyme mycothiol S-conjugate amidase to produce GlcN-Ins and AcCySR, a mercapturic acid, which is excreted from the cell; in MSR R is derived from the electrophile.

The biosynthesis of MSH has been identified as involving five steps: (1) formation of GlcNAc-Ins-P; (2) dephosphorylation of GlcNAc-Ins-P to produce GlcNAc-Ins; (3) deacetylation of GlcNAc-Ins to produce GlcN-Ins; (4) ligation of GlcN-Ins to Cys to produce Cys-GlcN-Ins; (5) acetylation of Cys-GlcN-Ins by acetyl-CoA to produce MSH. The enzymes catalyzing steps 1 (MshA), 3 (MshB), 4 (MshC) and 5 (MshA) are encoded by the genes designated mshA, mshB, mshC, and mshD, and these four genes have been identified. The mshA2 gene encoding the enzyme catalyzing step 2 has not yet been identified.

The structure of mycothiol, 1-D-myo-inosityl 2-(N-acetyl-L-cysteinyl)amido-2-deoxy-α-D-glucopyranoside, alternatively named 1-O-[2-[[(2R)-2-(acetylamino)-3-mercapto-1-oxopropyl]amino]-2-deoxy-α-D-glucopyranosyl]-D-myo-inositol, (AcCys-GlcN-Ins), makes it resistant to heavy-metal-catalyzed autoxidation and it appears to have functions analogous to those of glutathione. A mycothiol-dependent formaldehyde dehydrogenase has been identified. Mycobacterium smegmatis mutants defective in MSH biosynthesis exhibit enhanced sensitivity to hydrogen peroxide and modified sensitivity to antibiotics. Alkylating agents are detoxified by mycothiol and the resulting S-conjugates cleaved by an amidase to produce the N-acetylcysteine derivative (mercapturic acid), which is excreted from the cell. A mycothiol disulfide reductase maintains mycothiol in the reduced state.

Therefore, there is a need in the art for methods and compounds useful for investigation of the details of the metabolism of mycothiol and comparison with the established roles for the metabolism of glutathione and for identification of as yet unidentified biosynthesis genes.

Antibiotic resistance of pathogenic bacteria, including pathogenic actinomycetes, such as M. tuberculosis, is a well-known problem faced by medical practitioners in treatment of bacterial diseases. Therefore, there is a need in the art for new antibiotics, drugs and vaccines and for screening techniques to discover antibiotics, drugs and vaccines effective to treat or prevent bacterial infections in humans and in other mammals, such as domestic and farm animals.

### SUMMARY OF THE INVENTION

The present invention relates to identification of inhibitors of MshC, MshD and MshA, enzymes involved in the mycothiol biosynthesis pathway and provides methods utilizing such enzymes.

In one embodiment, the invention provides a method for identifying an inhibitor of cysteine:glucosaminyl inositol ligase (MshC). The method includes contacting a candidate compound with a cysteine:glucosaminyl inositol ligase in the presence of cysteine and a glucosaminyl inositol or a derivative thereof, under suitable conditions, and determining the presence or absence of ligation of the cysteine to the glucosaminyl inositol or derivative thereof. In the embodiment of the invention, a substantial absence of the ligation is indicative of a candidate compound that inhibits activity of the ligase. In another embodiment, the invention provides an inhibitor identified by the method.

In another embodiment, the invention provides a method for decreasing the virulence of a pathogenic cysteine:glucosaminyl inositol ligase-producing bacterium in mammalian cells. The method includes introducing an inhibitor of cysteine:glucosaminyl inositol ligase activity into the bacterium and observing the effect on the activity of the ligase. Where the intracellular presence of the inhibitor decreases activity of the ligase, mycothiol biosynthesis by the bacterium is also decreased, as compared with untreated control bacterium.

In another embodiment, the invention provides a method for increasing sensitivity of a pathogenic cysteine:glucosaminyl inositol ligase-producing bacterium in mammalian cells to an antibiotic. The method includes introducing an inhibitor of cysteine:glucosaminyl inositol ligase activity into the bacterium. The intracellular presence of the inhibitor decreases activity of the ligase, thereby decreasing mycothiol biosynthesis by the bacterium in said mammalian cells as compared with untreated control bacterium so as to increase sensitivity of the bacterium to an antibiotic.

The invention also provides a method for inhibiting growth of a glucosaminyl inositol-producing bacterium in a mammal. The method includes administering an effective amount of an inhibitor of intracellular cysteine:glucosaminyl inositol ligase to the mammal, thereby inhibiting growth of the bacterium in the mammal.

The invention also provides an inhibitor of MshC, which is identified as NTF1836. Also provided are homologs of NTF1836 as provided in Table 9 and Table 10.

In another embodiment, the invention provides a method for identifying an inhibitor of acetyl-CoA:cysteinyl glucosaminyl inositol (acetyl-CoA:Cys-GlcN-Ins) acetyltransferase (MshD). The method includes contacting a candidate compound with an acetyl-CoA:Cys-GlcN-Ins acetyltransferase in the presence of an acetyl-CoA and cysteinyl glucosaminyl inositol (Cys-GlcN-Ins) or a derivative thereof, under suitable conditions and determining the presence or absence of a transfer of acetyl to the Cys-GlcN-Ins or a derivative thereof. In the embodiment of the invention, the substantial absence of a transfer of acetyl is indicative of a candidate compound that inhibits activity of the acetyltransferase. In another embodiment, the invention provides an inhibitor identified by the method.

In another embodiment, the invention provides a method for increasing sensitivity of a pathogenic acetyl-CoA:Cys-GlcN-Ins acetyltransferase-producing bacterium in mammalian cells to an antibiotic. The method includes introducing an inhibitor of endogenous bacterial acetyltransferase activity into the bacterium, where the intracellular presence of the inhibitor decreases activity of the acetyltransferase. Such a decrease in activity also decreases mycothiol biosynthesis by the bacterium in said mammalian cells as compared with untreated control bacterium so as to increase sensitivity of the bacterium to an antibiotic.

In another embodiment, the invention provides a method for inhibiting growth of an acetyl-CoA:Cys-GlcN-Ins-producing bacterium in a mammal. The method includes administering to the mammal an effective amount of an inhibitor of intracellular acetyl-CoA:Cys-GlcN-Ins acetyltransferase, thereby inhibiting growth of the bacterium in the mammal.

In another embodiment, the invention provides a method for identifying an inhibitor of MshA glycosyltransferase (MshA). The method includes contacting a candidate compound with a mycothiol-producing bacterium under suitable conditions, and determining the presence or absence of 1D-myo-inosityl 2-acetamido-2-deoxy-α-D-glucopyranoside, alternatively named 1-O-(2-acetamido-2-deoxy-α-D-glucopyranosyl)-D-myo-inositol, (GlcNAc-Ins) within the mycothiol-producing bacterium. A substantial absence of GlcNAc-Ins within the bacterium is indicative of a compound that inhibits activity of the glycosyltransferase. In another embodiment, the invention provides an inhibitor identified by the method.

The invention also provides a method for inhibiting growth of a Gram-positive bacterium and/or a GlcNAc-Ins-producing bacterium in a subject. The method includes administering an effective amount of an inhibitor of intracellular MshA glycosyltransferase to the mammal. Such administration inhibits growth of the bacterium in the mammal. Exemplary Gram-positive bacteria include, but are not limited to, Staphylococcus aureus, Staphylococcus spp., Enterococcusfaecalis, Enterococcus spp., and Streptococcus spp.

In another embodiment, the invention provides a method for identifying an inhibitor of mycothiol biosynthesis. The method includes contacting a candidate compound for inhibition of MshC, MshD or MshA or a combination thereof with a mycothiol-producing bacterium under suitable conditions, and determining the presence or absence of mycothiol within the bacterium. A substantial absence of mycothiol within the bacterium is indicative of a compound that inhibits activity of the MshC, MshD or MshA and therefore inhibits mycothiol biosynthesis. In another embodiment, the invention provides an inhibitor identified by the method.

### BRIEF DESCRIPTION OF THE DRAWINGS

FIG. 1 is a graphical diagram showing growth of M. tuberculosis in the presence of NTF1836.

FIG. 2 is a graphical diagram showing growth of M. tuberculosis in the presence of NTF1836.

FIG. 3 is a pictorial diagram showing a reaction catalyzed by MshC with structures for GlcN-Ins and Cys-GlcN-Ins.

FIG. 4 is a pictorial diagram showing a restriction map of the pACE expression vector containing cloned M tuberculosis mshC (Rv2130c).

FIG. 5 is a graphical diagram showing progress of color reaction for samples of histidine buffer containing 100 μM Cys and 50 μM GlcN-Ins: O, BLANK, no additions; , 6 μM phosphate; ▴, 6 μM phosphate plus 0.1 mM ATP; +, 6 μM phosphate plus 0.1 mM ATP with citrate addition after 2 min.

FIG. 6 is a graphical diagram showing a standard curve for phosphate (O) and pyrophosphate (□) prepared in reaction mix without MshC and measured in 96-well microtitre plates. The line represents the least squares fit to all of the data with an average deviation of ±6%.

FIG. 7 is a graphical diagram showing time dependence of phosphate production in the coupled enzyme assay in the presence of 2 μg MshC (□), 1 μg MshC (O), and 1 μg MshC plus 1.6 mM of the MshC inhibitor cysteamine (Δ). Error bars represent standard deviation of quadruplicate determinations were larger than the symbol.

FIG. 8 is a graphical diagram showing an increase in fluorescence divided by A₆₀₀ after versus time following the addition of 50 μM mBCl to cells after growth of various strains in medium for 48 h in microplate wells.

FIG. 9 is a graphical diagram showing an increase in fluorescence upon treatment with mBCl divided by A₆₀₀ versus MSH content for various mutants as determined by HPLC following growth for 48 h in microplate wells.

FIG. 10 is a graphical diagram showing results from an assay of acceptor substrates with UDP-GlcNAc and dialyzed unfractionated crude extract of M. smegmatis mc²155.

FIG. 11 is a graphical diagram showing products as a function of time in the reaction of UDP-GlcNAc with D,L-Ins-1-P catalyzed by extracts of M. smegmatis mshB mutant Myco504.

FIG. 12 is a graphical diagram showing growth of mycobacterial strains mc²155 (100% MSH), Myco504 (5% MSH), and 49 (0% MSH) in the absence of INH.

FIG. 13 is a graphical diagram showing growth of mycobacterial strains as in FIG. 12, but in the presence of 10 μg per ml INH.

FIG. 14 is a graphical diagram showing A₆₀₀ value after 78 h of growth in 7H9 medium containing 2, 6 or 10 μg per ml INH. The initial A₆₀₀ value was 0.04 in each case.

FIG. 15 is a pictorial diagram showing mycothiol biosynthesis and intermediates. Cell extracts prepared in hot aqueous acetonitrile to inactivate and precipitate enzymes. After removal of acetonitrile samples are fluorescently labeled with AccQFluor before (GlcN-Ins determination) and after treatment with cloned MshB (GlcN-Ins+GlcNAc-Ins determination). Separate samples are labeled with mBBr during extraction in hot aqueous acetonitrile to preferentially label thiols (Cys-GlcN-Ins and MSH). HPLC analysis of the mBBr and AccQFluor labeled samples provides sensitive quantitative analysis for the biosynthetic intermediates and other cellular thiols. These analyses utilize standards and enzymes unique to our laboratory and serve to identify the inhibited step in the pathway.

FIG. 16 is a graphical diagram showing results from HPLC chromatography of substrates with varying numbers of phosphate residues using tetrabutylammonim ion pairing.

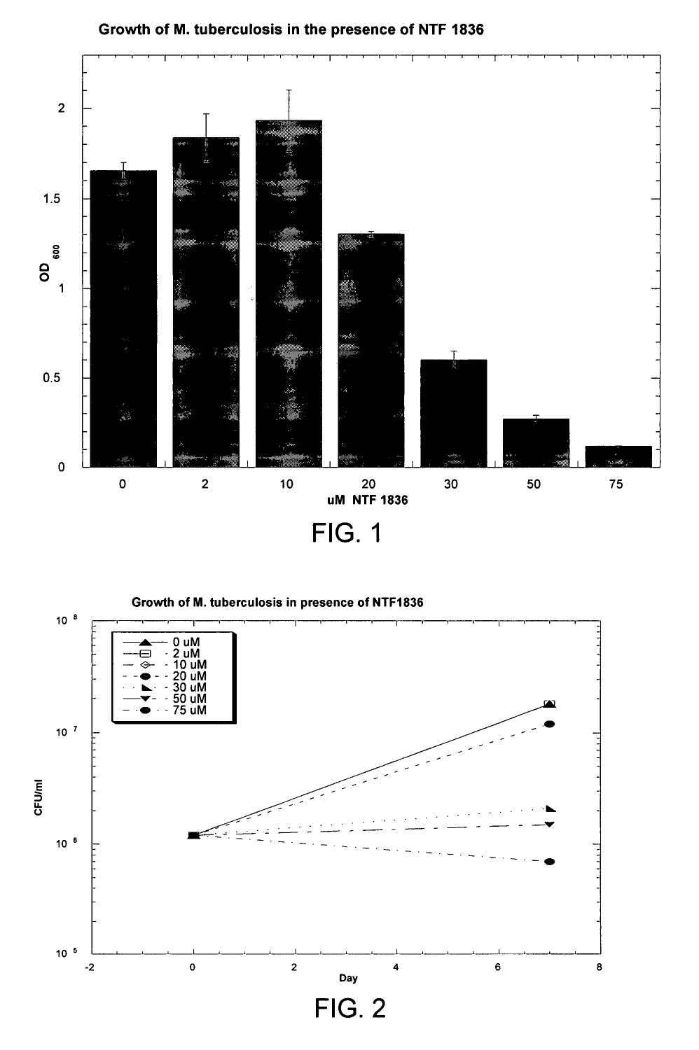

### DETAILED DESCRIPTION OF THE INVENTION

Mycothiol (1D-myo-inosityl 2-(N-acetylcysteinyl)amido-2-deoxy-I-D-glucopyranoside, alternatively named 1-O -[2-[[(2R)-2-(acetylamino)-3-mercapto-1-oxopropyl]amino]-2-deoxy-α-D-glucopyranosyl]-D-myo-inositol) (MSH) is present in a variety of actinomycetes and plays an essential role in a pathway of detoxification in such bacteria. Mycothiol is comprised of N-acetylcysteine (AcCys) amide linked to 1-O-(2-amino-2-deoxy-α-D-glucopyranosyl)-D-myo-inositol (GlcN-Ins) and is the major thiol produced by most actinomycetes. In the mycothiol-dependent detoxification process in actinomycetes, an alkylating agent is converted to a S-conjugate of mycothiol, the latter is cleaved to release a mercapturic acid, and the mercapturic acid is excreted from the cell (Newton et al. (2000a) Biochemistry 39:10739-10746).

Stopping the production of MSH should eliminate the MSH-dependent protective mechanisms and this makes the enzymes of mycothiol biosynthesis of special interest. The pathway of mycothiol biosynthesis involves at least five enzymes, which are designated MshA, MshA2, MshB, MshC and MshD. By the methods of the present invention, inhibitors of these enzymes can be identified for use in methods of drug discovery and disease control. MshB was previously identified and disclosed in U.S. application Ser. No. 10/297,512, filed Dec. 6, 2002, hereby incorporated by reference in its entirety, which is a national stage application of PCT/US01/19091, filed Jun. 14, 2001, which claims priority to U.S. Provisional Application 60/211,612, filed Jun. 14, 2000.

The ligase, MshC, is essential for production of MSH in Mycobacterium smegmatis but not for its growth. However, for Mycobacterium tuberculosis the mshC gene has been shown by targeted disruption and by high density mutagenesis to be essential for in vitro growth. It is therefore a potential target for drugs to treat tuberculosis. It appears likely that MshC will prove to be a drugable target since it is a homolog of cysteinyl-tRNA synthetase and considerable evidence indicates that tRNA synthetases are viable drug targets. Identifying inhibitors of MshC is therefore important to obtain leads for drug development. In addition, availability of inhibitors capable of blocking MSH production would provide a powerful tool for elucidation of the biological functions of MSH, not only in mycobacteria but in the diverse variety of actinomycetes that produce mycothiol.

A family of purified cysteine:glucosaminyl inositol ligase (MshC) polypeptides with catalytic ligase activity for glucosaminyl inositol (1-O-(2-amino-2-deoxy-α-D-glucopyranosyl)-D-myo-inositol; GlcN-Ins) and cysteine or a cysteine derivative is disclosed by co-pending PCT Application No. PCT/US03/11539, incorporated herein by reference. The members of the family of ligases catalyze ligation of cysteine to a glucosaminyl inositol or a derivative thereof. For example, the ligase catalyzes ATP-dependent ligation of L-cysteine to 1-O-(2-amino-2-deoxy-α-D-glucopyranosyl)-D-myo-inositol. In one embodiment, the glucosaminyl inositol is a precursor of mycothiol (e.g., in a mycothiol producing bacterium).

Additionally, there is provided a family of purified acetyl-CoA:cysteinyl glucosaminyl inositol (acetyl-CoA:Cys-GlcN-Ins) acetyltransferase (MshD) polypeptides with acetyltransferase activity for cysteinyl glucosaminyl inositol and acetyl-CoA, which are also disclosed by co-pending PCT Application No. PCT/US03/11539. The members of the family of acetyltransferases catalyze transfer of an acetyl group to a Cys-GlcN-Ins or derivative thereof, resulting in the production of mycothiol (AcCys-GlcN-Ins).

Additionally, a family of purified MshA glycosyltransferase (MshA) polypeptides with glycosyltransferase activity are also disclosed by co-pending PCT Application No. PCT/US03/11539. The members of the family of acetyltransferases catalyze production of 1-O-(2-acetamido-2-deoxy-α-D-glucopyranosyl)-D-myo-inositol 3-phosphate (GlcNAc-Ins-P) which is converted to 1-O-(2-acetamido-2-deoxy-α-D-glucopyranosyl)-D-myo-inositol (GlcNAc-Ins) by the phosphatase MshA2. In one embodiment, the GlcNAc-Ins is a precursor of mycothiol (e.g., in a mycothiol producing bacterium).

The methods described herein further elaborate the pathway involved in MSH biosynthesis. The invention demonstrates that GlcNAc-Ins-P and GlcNAc-Ins are intracellular mycothiol precursors and are formed by activity of MshA and MshA2, respectively. This conversion defines the initial steps in mycothiol biosynthesis. Further, the invention demonstrates that GlcN-Ins is an intracellular MSH component in M. smegmatis and is converted to Cys-GlcN-Ins by Cys:GlcN-Ins ligase (MshC). This conversion defines the penultimate step in MSH biosynthesis. Additionally, the invention demonstrates that Cys-GlcN-Ins is a precursor to mycothiol and is converted to mycothiol by acetyl-CoA:Cys-GlcN-Ins acetyltransferase (MshD) activity. This conversion defines the final step in MSH biosynthesis.

A member of the family of polypeptide ligases shown to be responsible for ATP-dependent ligation of L-cysteine to GlcN-Ins to form Cys-GlcN-Ins has been cloned from M. tuberculosis genomic sequence and corresponds to an open reading frame designated Rv2130c and misidentified as a probable cysS2, cysteinyl-tRNA synthetase (GenBank Accession # NP_{—}216646)(Cole, et al. (1998) Nature 393:537-544). The nucleic acid sequence encoding this protein corresponds to nucleic acids 2391213-2392457 of the M. tuberculosis genome encoding a protein of 414 amino acid residues. A BLAST search with the M. tuberculosis MshC sequence on GenBank revealed additional homologs in Corynebacterium striatum (Accession # AAG03366) and Streptomyces coelicolor (Accession # CAC36366). Orthologs of M. tuberculosis MshC were also found at the Sanger Centre in M. leprae (82% identity, S. T. Cole et al. (2001) Nature 409, 1007), M. bovis (96% identity; website Sanger.org/Projects/M_{—}bovis), and Corynebacterium diphtheriae (54% identity; website Sanger.org/Projects/C_{—}diphtheriae), and at TIGR in M. avium (81% identity). All of these organisms belong to genera of bacteria that have been shown to produce MSH (Newton et al., (1996) J. Bacteriol. 178, 1990-1995). This sequence homology indicates that MSH biosynthesis in these organisms utilizes a GlcN-Ins ligase (MshC) in the same manner as that described here for M. smegmatis.

A member of the family of polypeptide acetyltransferases shown to be responsible for acetylation of Cys-GlcN-Ins to form mycothiol has been cloned from M. tuberculosis genomic sequence and corresponds to an open reading frame designated Rv0819. The nucleic acid sequence encoding this protein corresponds to nucleic acids 911736-912680 of the M. tuberculosis genome encoding a protein of 315 amino acid residues. Sequence searches with the M. smegmatis mshD gene revealed orthologs in other actinomycetes including M. tuberculosis H37Rv. The M. tuberculosis gene (Rv0819) was cloned, expressed in E. coli, and shown to code for mycothiol synthase activity.

A member of the family of polypeptide glycosyltransferases shown to be required for formation of GlcNAc-Ins via the intermediate GlcNAc-Ins-P has been identified by gene disruption in the M. smegmatis genomic sequence and corresponds to an open reading frame designated MSMEG_{—}0933. The homolog in the M. tuberculosis H37Rv genome is designated Rv0486 and the nucleic acid sequence encoding this protein corresponds to nucleic acids 575346-576788 encoding a protein of 480 amino acid residues.

Members of the mycothiol biosynthesis families of enzymes are formed in vivo by bacteria as part of a mycothiol biosynthesis pathway, most usually in bacteria characterized by intracellular production of mycothiol. Additional bacteria from which the mycothiol biosynthesis polypeptides can be obtained include actinomycetes, such as M. smegmatis, M. tuberculosis, M. leprae, M. bovis, M. intracellulare, M. africanum, M. marinarum, M. chelonai, Corynebacterium diphtheria, Actinomycetes israelii, M. avium complex (MAC) (Holzman, in Tuberculosis ed. by Rom and Gary (Little, Brown, and Company, 1996) Chapter 56), M. ulcerans, M. abscessus, or M. scrofulaceum, and the like. Actinomycetes that can be used for this purpose include antibiotic-producing bacteria. Homologous non-mycobacterial ligase proteins can also be obtained from the antibiotic producers Streptomyces lincolnensis, Amycolatopsis mediterranei, Amycolatopsis orientalis, Streptomyces lavendulae, Streptomyces coelicolor, Streptomyces rochei, the polyketide erythromycin antibiotic producer Saccharopolyspora erythraea, Streptomyces violaceoruber Tu7, Streptomyces diastochromogens subsp. variabilicolor, and Streptomyces sp. OM-6519.

Inhibitors of the mycothiol biosynthesis ligases, acetyltransferases and glycosyltransferases are particularly well suited as antibiotics against mycothiol-producing bacteria since mycothiol production will cease in the absence of the intermediate products, GlcNAc-Ins or Cys-GlcN-Ins, produced by activity of the mycothiol biosynthesis enzymes. Accordingly, in one embodiment of the present invention, there are provided methods for identifying inhibitors of MshC, MshD, MshA and mycothiol biosynthesis.

As such, the invention provides a method for identifying an inhibitor of cysteine:glucosaminyl inositol ligase. The method includes contacting a candidate compound with a cysteine:glucosaminyl inositol ligase in the presence of cysteine and a glucosaminyl inositol or derivative thereof, under suitable conditions, and determining the presence or absence of ligation of cysteine to the glucosaminyl inositol or derivative thereof. For example, if the test compound is a putative inhibitor of ligase activity of the polypeptide ligase, the absence of ligated Cys-GlcN-Ins indicates the candidate compound is an inhibitor of the activity of the polypeptide as a ligase. Similarly, if the test compound is assayed as a putative inhibitor of MshC in mycothiol-producing bacteria, the presence of excess GlcN-Ins indicates that the candidate compound is an inhibitor of the activity of the ligase for linkage of cysteine or a cysteine derivative to a glucosaminyl inositol. On the other hand, in such assays, the presence of Cys-GlcN-Ins indicates that the test compound is not an inhibitor of MshC activity. In another embodiment, the candidate compound is contacted with a cysteine:glucosaminyl inositol ligase in the presence of cysteine, a glucosaminyl inositol or derivative thereof, ATP and pyrophosphatase, under suitable conditions, and detecting the resulting inorganic phosphate with a colorimetric or fluorometric assay known in the art.

As set forth above, the polypeptides can be derived from bacteria, including actinomycetes. In one embodiment of identifying an inhibitor of cysteine:glucosaminyl inositol ligase, the ligase is produced in an actinomycete. Additionally, the invention provides an inhibitor of cysteine:glucosaminyl inositol ligase identified by the method of the invention.

Similarly, the invention provides a method for identifying an inhibitor of acetyl-CoA:cysteinyl glucosaminyl inositol (acetyl-CoA:Cys-GlcN-Ins) acetyltransferase (MshD). The method includes contacting a candidate compound with an acetyl-CoA:Cys-GlcN-Ins acetyltransferase in the presence of a cysteinyl glucosaminyl inositol (Cys-GlcN-Ins) and acetyl-CoA, under suitable conditions and determining the presence or absence of a transfer of acetyl to the Cys-GlcN-Ins. In the embodiment of the invention, the substantial absence of a transfer of acetyl is indicative of a candidate compound that inhibits activity of the acetyltransferase. For example, if the test compound is a putative inhibitor of acetyltransferase activity of a polypeptide acetyltransferase, the absence of an acetylated Cys-GlcN-Ins (mycothiol) indicates the candidate compound is an inhibitor of the activity of the polypeptide as an acetyltransferase. Similarly, if the test compound is assayed as a putative inhibitor of MshD in mycothiol-producing bacteria, the presence of excess Cys-GlcN-Ins indicates that the candidate compound is an inhibitor of the activity of the acetyltransferase for linkage of an acetyl group to a Cys-GlcN-Ins. On the other hand, in such assays, the presence of mycothiol indicates that the test compound is not an inhibitor of MshD activity.

As set forth above, the polypeptides can be obtained from bacteria, including actinomycetes. In one embodiment of identifying an inhibitor of acetyl-CoA:Cys-GlcN-Ins acetyltransferase, the acetyltransferase is produced in an actinomycete. Additionally, the invention provides an inhibitor of acetyl-CoA:Cys-GlcN-Ins acetyltransferase identified by the method of the invention.

In still another related embodiment of the invention, a method for identifying an inhibitor of MshA glycosyltransferase (MshA) is provided. The method includes contacting a candidate compound with a mycothiol-producing bacterium under suitable conditions and determining the presence or absence of 1D-myo-inosityl 2-acetamido-2-deoxy-α-D-glucopyranoside (GlcNAc-Ins) within the mycothiol-producing bacterium. In the embodiment of the invention, the substantial absence of GlcNAc-Ins within the bacterium is indicative of a compound that inhibits activity of the glycosyltransferase. For example, if the test compound is a putative inhibitor of glycosyltransferase activity of the polypeptide glycosyltransferase, the absence of GlcNAc-Ins indicates the candidate compound is an inhibitor of the activity of the polypeptide as a glycosyltransferase. On the other hand, in such assays, the presence of GlcNAc-Ins indicates that the test compound is not an inhibitor of MshA activity.

As set forth above, the polypeptides can be obtained from bacteria, including actinomycetes. In one embodiment of identifying an inhibitor of MshA glycosyltransferase, the glycosyltransferase is produced in an actinomycete. Additionally, the invention provides an inhibitor of MshA glycosyltransferase identified by the method of the invention.

In still another embodiment, the invention provides a method for identifying an inhibitor of mycothiol biosynthesis. The method includes contacting a candidate compound with a mycothiol-producing bacterium, under suitable conditions, and determining the presence or absence of mycothiol within the mycothiol-producing bacterium. The substantial absence of mycothiol is indicative of a candidate compound that inhibits mycothiol biosynthesis. The inhibition of mycothiol biosynthesis can be by, but is not limited to, inhibition of cysteine:glucosaminyl inositol ligase, acetyl-CoA:Cys-GlcN-Ins acetyltransferase or MshA glycosyltransferase. Additionally, the excess or absence of intermediates of the mycothiol biosynthesis is indicative of an inhibitor of mycothiol biosynthesis. In another embodiment, the invention provides an inhibitor of mycothiol biosynthesis identified by the method.

In one embodiment, the mycothiol-producing bacterium of the method is an actinomycete. Additionally, the invention provides an inhibitor of mycothiol biosynthesis identified by the method of the invention.

In an alternative embodiment of the invention, methods are provided for decreasing the virulence in mammalian cells of a pathogenic MshC-producing, MshD-producing or MshA-producing bacterium, such as an actinomycete. By virulence is meant the relative power and degree of pathogenicity possessed by organisms to produce disease as measured by clinical symptoms particular to the disease under consideration. For example, the virulence of M. tuberculosis is measured with reference to the manifestation in an infected individual of the clinical symptoms recognized by a medical practitioner as indicative of tuberculosis. In the invention method for decreasing the virulence of pathogenic MshC-producing, MshD-producing or MshA-producing bacteria, an inhibitor of MshC, MshD or MshA (for example, one identified by the above-described screening method), respectively, is introduced into the bacterium.

Intracellular uptake of the inhibitor by the treated bacterium results in decreased activity of the enzyme, thereby decreasing mycothiol biosynthesis by the bacterium as compared with untreated control bacterium. Hence, the virulence of the treated bacterium is reduced. For example, for treatment of isolated mammalian cells, the introducing can comprise culturing the bacterium in the presence of the inhibitor. Alternatively, for treatment of mammalian cells contained in a living organism, the inhibitor may be administered systemically to the living organism. Pathogenic MshC-producing, MshD-producing or MshA-producing bacteria whose virulence can be reduced according to the invention methods include such actinomycetes as M. smegmatis, M. tuberculosis, M. leprae, M. bovis (particularly in bovine subjects), M. intracellulare, M. africanum, and M. marinarum. M. chelonai, Corynebacterium diphtheriae, Actinomyces israelii, M. avium complex (MAC), M. ulcerans, M. abscessus, M.scrofulaceum, and the like.

In one embodiment of the invention, a method for decreasing the virulence of a pathogenic cysteine:glucosaminyl inositol ligase-producing bacterium in mammalian cells is provided. Specifically, the method includes introducing an inhibitor of cysteine:glucosaminyl inositol ligase activity into the bacterium and observing the effect on the activity of the ligase. In the method of the invention, where the intracellular presence of the inhibitor causes a decrease in activity of the ligase, mycothiol biosynthesis by the bacterium is also decreased, as compared with untreated control bacterium.

The invention also provides a method for decreasing the virulence of a pathogenic acetyl-CoA:Cys-GlcN-Ins acetyltransferase-producing bacterium in mammalian cells. The method includes introducing an inhibitor of acetyl-CoA:Cys-GlcN-Ins acetyltransferase activity into the bacterium, where the intracellular presence of the inhibitor decreases activity of the acetyltransferase. Such a decrease also decreases mycothiol biosynthesis by the bacterium as compared with untreated control bacterium.

Similarly, the invention provides a method for decreasing the virulence of a pathogenic MshA glycosyltransferase-producing bacterium in mammalian cells. The method includes introducing an inhibitor of MshA glycosyltransferase activity into the bacterium. The intracellular presence of the inhibitor decreases activity of the glycosyltransferase, thereby decreasing mycothiol biosynthesis by the bacterium as compared with untreated control bacterium.

The inhibitors used in the invention methods for decreasing the virulence of a pathogenic MshC-producing, MshD-producing or MshA-producing bacterium may either inhibit intracellular production of the enzyme or inhibit intracellular catalytic activity of the enzyme. In one embodiment, the inhibitor inhibits intracellular production of mycothiol.

In another aspect, the invention provides methods of treating a Gram-positive bacterial invention in a subject by administering an inhibitor of the invention. Exemplary Gram-positive bacteria include, but are not limited to, Staphylococcus aureus, Staphylococcus spp., Enterococcusfaecalis, Enterococcus spp., and Streptococcus spp.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” ¢peripheral administration” and “administered peripherally” as used herein mean the administration of a compound, drug or other material other than directly into the central nervous system, such that it enters the subject's system and, thus, is subject to metabolism and other like processes, for example, subcutaneous administration.

In another embodiment, the present invention provides a method of ameliorating or treating a subject having an infection due to a microorganism with the subject inhibitors. As used herein, the term “ameliorating” or “treating” means that the clinical signs and/or the symptoms associated with the infection are lessened as a result of the actions performed. The signs or symptoms to be monitored will be characteristic of bacterial infection and will be well known to the skilled clinician, as will the methods for monitoring the signs and conditions.

Thus, the methods of the invention are useful for providing a means for practicing personalized medicine, wherein treatment is tailored to a subject based on the particular characteristics of the microbial infection in the subject. The method can be practiced, for example, by contacting a sample of cells from the subject with at least one inhibitor of the invention, wherein a decrease in signs or syptoms associated with the infection in the presence of the inhibitor as compared to the signs or syptoms associated with the infection in the absence of the inhibitor identifies the inhibitor as useful for treating the infection. The sample of cells examined according to the present method can be obtained from the subject to be treated, or can be cells of an established bacterial cell line of the same type as that of the infected subject.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

All methods may further include the step of bringing the active ingredient(s) into association with a pharmaceutically acceptable carrier, which constitutes one or more accessory ingredients. The term “pharmaceutically acceptable”, when used in reference to a carrier, is meant that the carrier, diluent or excipient must be compatible with the other ingredients of the formulation and not deleterious to the recipient thereof. Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The route of administration of a composition containing the inhibitors of the invention will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the inhibitor is a small organic molecule such as a steroidal alkaloid, it can be administered in a form that releases the active agent at the desired position in the body (e.g., the liver), or by injection into a blood vessel such that the inhibitor circulates to the target cells.

Exemplary routes of administration include, but are not limited to, orally or parenterally, such as intravenously, intramuscularly, subcutaneously, intraperitoneally, intrarectally, intracistemally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of infection, for example, intravenously or intra-arterially into a blood vessel.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the inhibitor of the invention to treat bacterial infection in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In various embodiments of the present invention there are provided inhibitors of the enzymes of mycothiol biosynthesis. These inhibitors may be identified, for example, by the screening method set forth above. For example, the inhibitor can be, but is not limited to a polypeptide, a polynucleotide or a small molecule. As disclosed herein, the screening methods of the invention provide the advantage that they can be adapted to high throughput analysis and, therefore, can be used to screen combinatorial libraries of test agents in order to identify those agents that can inhibit the enzymes of mycothiol biosynthesis. Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13 19, 1991; each of which is incorporated herein by reference); a peptide library (U.S. Pat. No. 5,264,563, which is incorporated herein by reference); a peptidomimetic library (Blondelle et al., Trends Anal. Chem. 14:83 92, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference); an oligosaccharide library (York et al., Carb. Res., 285:99 128, 1996; Liang et al., Science, 274:1520 1522, 1996; Ding et al., Adv. Expt. Med. Biol. 376:261 269, 1995; each of which is incorporated herein by reference); a lipoprotein library (de Kruif et al., FEBS Lett. 399:232 236, 1996, which is incorporated herein by reference); a glycoprotein or glycolipid library (Karaoglu et al., J. Cell Biol. 130:567 577, 1995, which is incorporated herein by reference); or a chemical library containing, for example, drugs or other pharmaceutical agents (Gordon et al., J. Med. Chem. 37:1385-1401, 1994; Ecker and Crooke, BioTechnology 13:351-360, 1995; each of which is incorporated herein by reference). Polynucleotides can be particularly useful as agents that can modulate a specific interaction of molecules because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

When adapted to high throughput analysis, the methods of the invention may include screenting of a plurality of test agents, which can be arranged in an array (e.g., an addressable array) on a solid support such as a microchip, on a glass slide, on a bead, or in a well, and the bacterium of interest (e.g., mycothiol-producing bacterium) can be contacted with the different test agents to identify one or more agents having desirable characteristics, including, for example, the ability to inhibit the enzymes of mycothiol biosynthesis.

An additional advantage of arranging the samples in an array, particularly an addressable array, is that an automated system can be used for adding or removing reagents from one or more of the samples at various times, or for adding different reagents to particular samples. In addition to the convenience of examining multiple test agents and/or samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays. In one embodiment, where inhibition of MshC is desired, the invention inhibitors may be derived from L-cysteine by replacing the carboxyl group with a moiety that binds the enzyme active site. Examples of the type of moiety that can be used to replace the carboxyl group in L-cysteine to form an inhibitor of the ligases are selected from moieties having the chemical structure CH₂OPO(OH)OR, wherein R is derived either from AMP or from a cyclitol bearing one or more branched or unbranched alkyl residues. In another embodiment where inhibition of MshC is desired, the invention inhibitors are derived from L-cysteine by replacing the carboxyl group therein with a moiety having the chemical structure CONHSO₂OR, wherein R is derived from AMP or R is a cyclitol bearing one or more branched or unbranched alkyl residues. Suitable alkyl residues for this purpose include, but are not limited to, those containing from 1 to 10 carbons, for example 2 to 8 carbons, 3 to 6 carbons, or 4 to 5 carbons.

In yet another embodiment according to the present invention, there are provided methods for increasing sensitivity to an antibiotic of a pathogenic MshC-producing, MshD-producing or MshA-producing bacterium.

In such a method for increasing sensitivity of a pathogenic cysteine:glucosaminyl inositol ligase (MshC)-producing bacterium in mammalian cells, an invention inhibitor of cysteine:glucosaminyl inositol ligase activity is introduced into the bacterium. The intracellular presence of the invention inhibitor in the bacterium decreases activity of the ligase, thereby decreasing mycothiol biosynthesis by the bacterium as compared with untreated control bacterium so as to increase sensitivity of the bacterium to an antibiotic. The inhibitor can inhibit intracellular production of the ligase or inhibit intracellular ligase activity of the ligase. In one embodiment, sensitivity of the bacterium to the antibiotic is increased while the bacterium is within a mammalian cell by a decreased activity of the ligase in the bacterium while contained within the host mammalian cell. The inhibitor can be introduced into the bacterium, for example, by culturing the bacterium in the presence of the inhibitor. Bacteria whose sensitivity to antibiotics can be increased by practice of the invention methods include such pathogenic bacteria as various actinomycetes. Specific examples of bacteria whose sensitivity to antibiotics can be increased by the invention methods include M. smegmatis, M. tuberculosis, M. leprae, M. bovis, M. intracellulare, M. africanum, M. marinarum. M. chelonai, Corynebacterium diphtheria, Actinomyces israeli, M. avium complex (MAC), M. ulcerans, M. abscessus, M. scrofulaceum, and the like. In another embodiment, the bacterium is an actinomycete and the inhibitor inhibits intracellular production of mycothiol.

Similarly, in a method for increasing sensitivity of a pathogenic acetyl-CoA:Cys-GlcN-Ins acetyltransferase (MshD)-producing bacterium in mammalian cells to an antibiotic, an inhibitor of acetyl-CoA:Cys-GlcN-Ins acetyltransferase activity is introduced into the bacterium. The intracellular presence of the inhibitor in the bacterium decreases activity of the acetyltransferase, thereby decreasing mycothiol biosynthesis by the bacterium as compared with untreated control bacterium so as to increase sensitivity of the bacterium to an antibiotic. The inhibitor can inhibit intracellular production of the acetyltransferase or inhibit intracellular activity of the acetyltransferase. In one embodiment, sensitivity of the bacterium to the antibiotic is increased while the bacterium is within a mammalian cell by a decreased activity of the acetyltransferase in the bacterium while contained within the host mammalian cell. The inhibitor can be introduced into the bacterium, for example, by culturing the bacterium in the presence of the inhibitor.

Additionally, in a method for increasing sensitivity of a pathogenic glycosyltransferase (MshA)-producing bacterium in mammalian cells to an antibiotic, an inhibitor of MshA glycosyltransferase activity is introduced into the bacterium. The intracellular presence of the inhibitor in the bacterium decreases activity of the glycosyltransferase, thereby decreasing mycothiol biosynthesis by the bacterium as compared with untreated control bacterium so as to increase sensitivity of the bacterium to an antibiotic. The inhibitor can inhibit intracellular production of the glycosyltransferase or inhibit intracellular activity of the glycosyltransferase. In one embodiment, sensitivity of the bacterium to the antibiotic is increased while the bacterium is within a mammalian cell by a decreased activity of the glycosyltransferase in the bacterium while contained within the host mammalian cell. The inhibitor can be introduced into the bacterium, for example, by culturing the bacterium in the presence of the inhibitor.

In still another embodiment, the invention provides a method for increasing sensitivity of a pathogenic mycothiol-producing bacterium in mammalian cells to an antibiotic, by introducing an inhibitor of endogenous bacterial mycothiol biosynthesis into the bacterium. The intracellular presence of the inhibitor in the bacterium decreases mycothiol biosynthesis by the bacterium as compared with untreated control bacterium so as to increase sensitivity of the bacterium to an antibiotic. The inhibitor can inhibit intracellular production of cysteine:glucosaminyl inositol ligase (MshC), acetyl-CoA:Cys-GlcN-Ins acetyltransferase (MshD) or glycosyltransferase (MshA) or can inhibit activity of the same. The inhibitor can be introduced into the bacterium, for example, by culturing the bacterium in the presence of the inhibitor.

Bacteria whose sensitivity to antibiotics can be increased by practice of the invention methods include such pathogenic bacteria as various actinomycetes. Specific examples of bacteria whose sensitivity to antibiotics can be increased by the invention methods include M. smegmatis, M. tuberculosis, M. leprae, M. bovis, M. intracellulare, M. africanum, M. marinarum. M. chelonai, Corynebacterium diphtheria, Actinomyces israeli, M. avium complex (MAC), M. ulcerans, M. abscessus, M. scrofulaceum, and the like. In another embodiment, the bacterium is an actinomycete and the inhibitor inhibits intracellular production of mycothiol.

In accordance with the above, the invention also provides a method of synthesizing mycothiol in vivo. By inserting mshA, mshB, mshC and mshD, the genes for the four enzymes of mycothiol production, together with a gene encoding a phosphatase active on GlcNAc-Ins-P, into a plasmid and inserting the plasmid into an organism, all four enzymes are expressed. With this expression, mycothiol is produced by the host cell. This method may be used to stimulate mycothiol production in the organism or increase existing mycothiol production. Such an increase of mycothiol within an organism serves to increase tolerance of the organism to antibiotics. Such an increase in antibiotic tolerance is useful to protect antibiotic-producing organisms from the toxic effects of the antibiotics they produce.

Therefore in an embodiment according to the present invention, there are provided methods for increasing production of antibiotic by antibiotic-producing bacteria by transforming the antibiotic-producing bacteria with a polynucleotide that increases intracellular mycothiol production by the bacteria in culture. The increase in intracellular production of mycothiol increases the production of antibiotic by the bacteria by increasing resistance of the bacteria to the antibiotic. Generally, in industrial applications where antibiotic is produced from bacteria for commercial purposes, the antibiotic-producing bacteria are cultured under conditions suitable for production of the antibiotic, and the antibiotic is recovered from the culture media.

In one embodiment, the compound that increases intracellular mycothiol production by the bacteria is expressed intracellularly by the bacteria. In one embodiment, the bacteria is actinomycetes. For example, the actinomycetes can be transformed with a polynucleotide, such as an expression vector, that encodes one or more enzymes involved in the mycothiol biosynthesis pathway and which produces mycothiol in culture. Recombinant expression of the polypeptides in cultured antibiotic-producing cells can be useful for increasing the resistance of the production cells to the toxic effect upon themselves of the antibiotics they produce. Thus, the level of antibiotics in the culture media can be increased without causing death of the production cells, thereby increasing the efficiency of industrial antibiotic production methods.

In yet another embodiment according to the present invention, there are provided live mutant actinomycetes, whose genomes comprise a modification in an endogenous enzyme of the mycothiol biosynthesis pathway and thereby reduce mycothiol synthesis. Appropriate modification of genes for mycothiol biosynthesis in mycobacteria can reduce their survival in mammalian macrophages. Modification of any one of the endogenous cysteine:glucosaminyl inositol ligase gene, acetyl-CoA:Cys-GlcN-Ins acetyltransferase gene or MshA glycosyltransferase gene can reduce function of an endogenous cysteine:glucosaminyl inositol ligase, acetyl-CoA:Cys-GlcN-Ins acetyltransferase or MshA glycosyltransferase, respectively, while cell surface proteins and lipids should be substantially unaffected. As a result, invention live mutant actinomycetes exhibit the phenotype of transient survival in mammalian white blood cells, such as murine or human white blood cells, for an immune response raising period of time. Such genetically engineered live mutant actinomycetes will survive in mammalian white blood cells for a period of time from 1 to 30 days, for example from 4 to 25 days or from 5 to 20 days, but in no event for more than 30 days. Due to lack of intracellular cysteine:glucosaminyl inositol ligase, acetyl-CoA:Cys-GlcN-Ins acetyltransferase or MshA glycosyltransferase, the invention live mutant bacterium will fail to produce sufficient mycothiol. Hence, the mutant live bacterium is unable to survive the oxidative stress inherent in the intracellular environment of mammalian white blood cells long enough to establish infection in the cells or to establish infection in an immunocompetent mammal containing such white blood cells. In one embodiment, the live mutant contains a modification of the acetyl-CoA:Cys-GlcN-Ins acetyltransferase gene or the MshA glycosyltransferase gene and the resulting mutant is resistant to isoniazid.

Thus, the invention live mutant actinomycetes possess a combination of features desired for a vaccine effective in mammals against infection by such pathogenic actinomycetes as M. smegmatis, M. tuberculosis, M. leprae, M. bovis, M. intracellulare, M. africanum, M. marinarum. M. chelonai, Corynebacterium diphtheria, Actinomycetes israeli, M. avium complex (MAC), M. ulcerans, M. abscessus, M. scrofulaceum, and the like. An individual (e.g., an animal, such as a mouse, a farm animal or a human) to which the live mutant is administered according to a protocol appropriate for inducing a protective immune response and who has not previously been infected by the counterpart wild type will mount an immune response to the vaccine, for example an immune response sufficient to protect the individual against future infection by the corresponding wild type live pathogen. Alternatively, the invention live mutant actinomycetes are useful as a research tool to investigate the properties desirable in a live mutant vaccine.

The invention also provides a method for inhibiting growth of Cys-GlcN-Ins-producing bacterium, acetyl-CoA:Cys-GlcN-Ins acetyltransferase-producing bacterium or GlcNAc-Ins-producing bacterium. In the method reducing the growth of Cys-GlcN-Ins-producing bacterium, an inhibitor of intracellular cysteine:glucosaminyl inositol ligase is administered to the mammal. In the method reducing the growth of acetyl-CoA:Cys-GlcN-Ins acetyltransferase-producing bacterium, an inhibitor of intracellular acetyl-CoA:Cys-GlcN-Ins acetyltransferase is administered to the mammal. Similarly, in the method reducing the growth of GlcNAc-Ins-producing bacterium, an inhibitor of intracellular MshA glycosyltransferase is administered to the mammal. Such administration of an inhibitor of cysteine:glucosaminyl inositol ligase, acetyl-CoA:Cys-GlcN-Ins acetyltransferase or MshA glycosyltransferase will inhibit growth of the bacterium in the mammal. In one embodiment, the bacterium is a mycothiol-producing bacterium. Bacteria whose growth can be inhibited by the practice of the invention methods utilizing such inhibitors can include such pathogenic bacteria as various actinomycetes. Specific examples of bacteria whose growth can be inhibited by the invention methods include M. smegmatis, M. tuberculosis, M. leprae, M. bovis, M. intracellulare, M. africanum, M. marinarum. M. chelonai, Corynebacterium diphtheria, Actinomyces israeli, M. avium complex (MAC), M. ulcerans, M. abscessus, M. scrofulaceum, and the like.

The invention also provides antibodies that are specifically reactive with mycothiol biosynthesis enzyme polypeptides or fragments thereof. Such antibodies can be used as research tools to aid in isolation of mycothiol biosynthesis enzymes such as MshC, MshD or MshA.

The invention also provides antibodies that consist essentially of pooled monoclonal antibodies with different epitopic specificities, as well as distinct monoclonal antibody preparations are provided. Monoclonal antibodies are made from antigen-containing fragments of the protein by methods well known in the art (Kohler, et al., Nature, 256:495, 1975; Current Protocols in Molecular Biology, Ausubel, et al., ed., 1989). Monoclonal antibodies specific for MshC polypeptide, MshD polypeptide or MshA polypeptide can be selected, for example, by screening for hybridoma culture supernatants that react with the MshC polypeptide, MshD polypeptide or MshA polypeptide, but do not react with other bacterial ligases, acetyltransferases or glycosyltransferases, respectively.

Additionally, the invention provides antibodies that consist essentially of pooled monoclonal antibodies with different epitopic specificities, as well as distinct monoclonal antibody preparations are provided. Monoclonal antibodies are made from antigen containing fragments of a protein by methods well known in the art (Kohler, et al., Nature, 256:495, 1975; Current Protocols in Molecular Biology, Ausubel, et al., ed., 1989).

The term “antibody” as used in this invention includes intact molecules as well as fragments thereof, such as Fab, F(ab′)₂ and Fv, which are capable of binding the epitopic determinant. These antibody fragments retain some ability to selectively bind with its antigen or receptor and are defined as follows:
(1) Fab, the fragment which contains a monovalent antigen-binding fragment of an antibody molecule can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)₂ is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New. York (1988), incorporated herein by reference).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

Antibodies that bind to a MshC polypeptide, MshD polypeptide or MshA polypeptide of the invention can be prepared using an intact polypeptide or fragments containing small peptides of interest as the immunizing antigen. The polypeptide or a peptide used to immunize an animal can be derived from translated cDNA or chemical synthesis and can be conjugated to a carrier protein, if desired. Such commonly used carriers, which are chemically coupled to the peptide, include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated herein by reference).

It is also possible to use anti-idiotype technology to produce monoclonal antibodies that mimic an epitope. For example, an anti-idiotypic monoclonal antibody made to a first monoclonal antibody will have a binding domain in the hypervariable region that is the “image” of the epitope bound by the first monoclonal antibody.

The present invention also features transgenic non-human organisms, e.g. live mutant actinomycetes, which either express a heterologous mshC, mshD or mshA gene, or in which expression of their own mshC, mshD or mshA gene is disrupted. In addition to the other utilities of such organisms disclosed herein, such a transgenic organism with a disrupted mshC gene has utility for overproduction of glucosaminyl inositol needed for screening (particularly high throughput screening) for compounds that inhibit cysteine:glucosaminyl inositol ligase activity in mycothiol-producing bacteria. Similarly, a transgenic organism with a disrupted mshD gene has utility for overproduction of Cys-GlcN-Ins.

Yet another aspect of the invention pertains to a peptidomimetic that binds to or interferes with a MshC polypeptide, MshD polypeptide or MshA polypeptide and inhibits its respective activity. For example, a peptidomimetic that binds to or interferes with a MshC polypeptide can inhibit binding to or linkage of substrate cysteine to glucosaminyl inositol or a derivative thereof. Non-hydrolyzable peptide analogs of such residues can be generated using, for example, benzodiazepine, azepine, substituted gama-lactam rings, keto-methylene pseudopeptides, beta-turn dipeptide cores, or beta-aminoalcohols. Similarly, a peptidomimetic that binds to or interferes with a MshD polypeptide can inhibit binding to or linkage of acetyl to substrate Cys-GlcN-Ins or a derivative thereof. An exemplary peptidomimetic binds to or interferes with a MshA polypeptide and can inhibit production of GlcNAc-Ins.

Other features and advantages of the invention will be apparent from the detailed description herein, and from the claims. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or anti-sense) and double-stranded polynucleotides.

As used herein, the terms “gene,” “recombinant gene” and “gene construct” refer to a nucleic acid comprising an open reading frame encoding a cysteine:glucosaminyl inositol ligase, acetyl-CoA:Cys-GlcN-Ins acetyltransferase or MshA glycosyltransferase, including both exon and (optionally) intron sequences. The term “intron” refers to a DNA sequence present in a given cysteine:glucosaminyl inositol ligase, acetyl-CoA:Cys-GlcN-Ins acetyltransferase or MshA glycosyltransferase gene that is not translated into protein and is generally found between exons.

The identification of the mycothiol biosynthesis genes establishes the basis for production of MSH biosynthesis gene knockouts in M. tuberculosis. Such knockouts can be used in determining the role of MSH in the virulence of M. tuberculosis

The following examples are intended to illustrate but not limit the invention.

### Example 1

### Assay for MshC Activity

Assay of MshC activity has thus far been accomplished by monitoring the production of Cys-GlcN-Ins; the thiol group of Cys-GlcN-Ins is labeled with monobromobimane (mBBr) to produce the highly fluorescent bimane derivative CySmB-GlcN-Ins which is analyzed with high sensitivity by high performance liquid chromatography (HPLC) and fluorescence detection. However, a simpler, more rapid analysis was desirable, especially for high throughput screening of potential inhibitors. The objective of the present work was to develop and test a spectrophotometric assay for MshC activity based on the determination of pyrophosphate produced in the reaction. u

has been often used to convert pyrophosphate to two equivalents of phosphate that is then detected by various techniques. In the present study, a coupled enzyme assay for MshC was developed using pyrophosphatase to generate phosphate. For sensitivity and ease of analysis, the phosphate is quantified by colorimetric measurement of the complex of phosphomolybdate with malachite green.

Reagents purchased commercially were of the highest purity available. Ammonium molybdate tetrahydrate, ATP, L-cysteine, histidine, malachite green hydrochloride, malachite green oxalate, 2-mercaptoethanol, E. coli inorganic pyrophosphatase, tetrasodium pyrophosphate, and Triton X-100 were from Sigma. High-purity dithiothreitol and hygromycin B were from Calbiochem, spectragrade dimethylsulfoxide was from Aldrich, and monobromobimane (mBBr) was from Molecular Probes. Middlebrook 7H9 broth and OADC enrichment were from Becton Dickinson. All other reagents were of reagent or higher grade and were from Fisher.

GlcN-Ins was prepared by enzymatic cleavage of the mBBr derivative of mycothiol derived from Mycobacterium smegmatis by a modified version of the published protocol (Newton, et al., A novel mycothiol-dependent detoxification pathway in mycobacteria involving mycothiol S-conjugate amidase, Biochemistry (2000) 39, 10739-10746). Forty one-liter cultures of M. smegmatis mc^{2 155} in Middlebrook 7H9 medium with 0.05% Tween 80 and 1% glucose were grown to A₆₀₀=3.0 and harvested by centrifugation to generate 200 g wet weight of cells. The frozen cells were suspended in 1000 ml of warm (˜60° C.) 50% aqueous acetonitrile containing 0.74 mM mBBr with a Tissuemizer (Tekmar) and the suspension heated at 60° C. for 15 min to lyse the cells. The suspension was centrifuged (30 min, 8,000 g); the supernatant was retained and the pellet washed with warm 50% aqueous acetonitrile lackmg mBBr. The supernatants were combined, reduced to a volume of 300 ml on a rotary evaporator, and centrifuged (30 min, 30,000 g) to remove residual solid material. The supernatant was divided into four equal portions, each of which was applied to a 20 g Sep Pak C-18 column (Waters) and eluted first with 100 ml 0.1% aqueous trifluoroacetic acid, then with 100 ml 10% methanol in 0.1% aqueous trifluoroacetic acid, and finally with 20% aqueous methanol in 0.1% aqueous trifluoroacetic acid. MSmB eluted in the 10% methanol fraction, was concentrated to dryness on a Savant SpeedVac, and was taken up in a minimal volume of water. The MSmB was purified by preparative HPLC on a 1.0×25 cm Vydac C-18 column (218TP 1022) which was eluted at 5 ml per min with a 0-20% methanol gradient over 50 min in 0.1% aqueous trifluoroacetic acid. The MSmB eluted at ˜20 min following a yellow contaminant which partly overlapped the MSmB peak. The combined fractions contained a total of 158 μmol of MSmB as assayed by HPLC (Koledin, et al., Identification of the mycothiol synthase gene (mshD) encoding the acetyltransferase producing mycothiol in actinomycetes, Arch. Microbiol. (2002) 178,33 1-337). Methanol and residual trifluoroacetic acid were removed by repeated drying on a Savant SpeedVac and resolubilization in water. The residue was dissolved in a minimal volume of water, analyzed by HPLC for MSmB content, and adjusted to ˜20 mM MSmB. To hydrolyze the MSmB to GlcN-Ins and AcCySmB, this solution was treated in two main batches with 0.5 μg mycothiol S-conjugate amidase (Steffek, et al., Characterization of Mycobacterium tuberculosis mycothiol S-conjugate amidase, Biochemistry (2003) 42, 12067-12076) per μmol MSmB at room temperature for 18 h at pH 7, the pH adjusted by periodic addition of 1 N NaOH. The mixtures were applied to 1 g SepPak C-18 columns and eluted with 0.05% aqueous trifluoroacetic acid. GlcN-Ins was monitored by HPLC analysis of the AccQ-Fluor derivative as previously described (Anderberg, et al., Mycothiol biosynthesis and metabolism: Cellular levels of potential intermediates in the biosynthesis and degradation of mycothiol, J. Biol. Chem. (1998) 273,3039 1-30397) and eluted at 1-5 ml. The AcCySmB and mycothiol S-conjugate amidase remained on the column during this solid phase extraction. A total of 106 μmol of GlcN-Ins was obtained as a 20 mM solution which was adjusted to pH 7 with NaOH.

Cloning of MshC in pACE. The MshC used in these studies was cloned from M. tuberculosis (Rv2 130c) and expressed in mshC deficient mutant strain I64 of M.smegmatis; the enzyme was purified as described below. The mshC/Rv2130c gene had been previously cloned in pRSETA into BamHI/HindIII sites under the T7 promoter (Sareen, et al., ATP-dependent L-cysteine:1D-myo-inosityl 2-amino-2-deoxy-a-D-glucopyranoside ligase, mycothiol biosynthesis enzyme MshC, is related to class I cysteinyl-tRNA synthetases, Biochemistry (2002) 41,6885-6890). The expression of the gene in E. coli on isopropyl β-D-thiogalactopyranoside induction showed that the protein was largely insoluble and became aggregated in the inclusion bodies. The expression was then attempted in M smegmatis under the acetamidase promoter of pACE [De Smet, et al., Alteration of a single amino acid residue reverses fosfomycin resistance of recombinant MurA from Mycobacteriurn tuberculosis, Microbiol. (1999) 145,3 177-3 184). To accomplish this the mshC gene in pRSETA (Sareen et al., 2002) was restriction digested with BamHI and Hind III and subcloned in the vector pSODIT-2 (De Smet, et al., 1999) at the same two respective sites. The gene was then cut from this vector with BamHI and ClaI and subcloned at the respective sites in pACE (De Smet, et al., 1999) (FIG. 4). The pACEmshC was used to electrotransform the M smegmatis I64 mutant, which is deficient in MshC activity (Rawat, et al., Mycothioldeficient Mycobacterium smegmatis mutants are hypersensitive to alkylating agents, free radicals and antibiotics, Antimicrob. Agents Chemother. (2002) 46,3348-3355). The electrocompetent cells of mutant I64 were made by repeated washing of the cells cultured to exponential phase (A₆₀₀=0.5) with sterile 10% glycerol. After electroporation at 1000 Ω, 25 μF, 2.5 kV, the cells were supplemented with one ml of 7H9 medium plus 1% glucose and shaken at 37° C. for 4 hours before plating onto Middlebrook 7H9 agar supplemented with 1% glucose and 75 μg per ml hygromycin. The M. smegmatis MshC mutant I64 complemented with M. tuberculosis mshC (Rv2130c) in pACE vector is hereafter denoted I64::pACEmshC.

Preparation of MshC. Strain I64::pACEmshC was grown in Middlebrook 7H9 medium supplemented with 0.05% Tween 80, 10% OADC and hygromycin (75 μg/ml) at 37° C. and 250 rpm. The culture was propagated on a large scale in the same media but with 1% glucose instead of 10% OADC. The induction was initiated at A₆₀₀=0.3 by centrifuging the cells at 8,000 g for 15 min and resuspending them in new media with 0.4% acetamide in place of glucose while maintaining the antibiotic selection pressure. After 28 h of cultivation at 37° C. with shaking (250 rpm), the cells were collected by centrifugation at 8,000 g for 15 min. The cell pellets, about 2.3 g per liter, were stored at −70° C. until used.

All purification steps were carried out at 4° C. in the presence of 3 mM 2-mercaptoethanol and 5 mM MgCl₂ unless stated otherwise. Protein concentration was determined by the method of Bradford (Bradford, et al., A rapid and sensitive method for the quantitation of microgram quantities of protein utilizing the principle of protein-dye binding, Anal. Biochem. (1976) 72,248-254). Twenty grams of I64::pACEmshC cells (wet weight) were suspended in 80 ml of extraction buffer (50 mM Hepes buffer, pH 7.5) containing 35 μM each of the protease inhibitors N-α-p-tosyl-L-phenylalanylchloromethyl ketone and N-α-p-tosyl-L-lysinechloromethyl ketone, both from Sigma. The cells were disrupted by ultrasonication (Branson Sonifier 200) in an ice bath. The cell debris was removed by centrifugation at 100,000 g for 1 h. Ammonium sulfate was added to 20% saturation on ice and the mixture allowed to stand for 2 h before centrifugation at 28,000 g for 30 min. Ammonium sulfate was added to the supernatant to 45% saturation and the mixture was stored overnight at 4° C. The precipitated proteins were pelleted by centrifugation at 28,000 g for 30 min. The pellet (4.8 g) was resusupended in 48 ml of the extraction buffer and the solution was desalted by passing it through a 3×23 cm Sephadex G-25 (Pharmacia) column preequilibrated with 50 mM Hepes, pH 7.5.

The G-25 eluent was applied on a DEAE 650-M (Toso Haas) column (5.2×10.6 cm) preequilibrated with 50 mM Hepes, pH 7.5. The enzyme eluted at 0.2 M NaCl in a linear gradient of 0-0.5 M NaCl in 15 column volumes of the buffer at 300 ml per h. The fractions containing the enzyme activity were combined (235 ml) and diluted to 480 ml with Milli-Q water to lower the salt concentration. The diluted solution was applied to a Bio-gel hydroxyapetite (HTP, BioRad) column (2.6×11.3 cm) pre-equilibrated with 10 mM potassium phosphate buffer pH 6.8 containing 100 mM NaCl. The bound proteins were eluted at 120 ml per h with a linear gradient from 10 mM phosphate/100 mM NaCl to 100 mM phosphate/0 mM NaCl in 15 column volumes. The active fractions were collected (76 ml) and peak activity fractions were analyzed for purity on 12.5% SDS-PAGE. High purity fractions were pooled, precipitated with 80% ammonium sulfate as a concentration step, and taken up in 50 mM Hepes buffer pH 7.5 for gel filtration chromatography on a Sephacryl-200 (Pharmacia) column (1.5×100 cm) at 10 ml/h in 50 mM Hepes, pH 7.5 and 150 mM NaCl. Active fractions were analyzed on SDS-PAGE and those of highest purity pooled, concentrated using 10 kD membrane filters (Amicon) and stored in 50% glycerol at −70° C. in 30 μL aliquots until used. With the activity of the crude extract defined at 100%, the overall yield of activity was only 1-3% of 50-80% pure enzyme (specific activity ˜150 nmol min⁻¹ mg⁻¹) as determined by SDS-PAGE. However, the protein was sufficiently pure for inhibitor screening after chromatography on Bio-gel HTP at which point the yield of activity was 5-10% and ATPase activity was absent. The MshC activity is maximal at pH 8.5 and 5-fold lower at pH 7.0; assay at pH 8.0 was chosen as a compromise between optimizing MshC activity and limiting the oxidation of cysteine with accompanying oxidation of dithiothreitol.

Coupled enzyme spectrophotometric assay. The following solutions were prepared as indicated: reagent A, 75 mM ammonium molybdate tetrahydrate in 4 N HCl stored at 4° C. for up to 6 months; reagent B, 1.5 mM malachite green hydrochloride in 3.06 M H₂SO₄ stored at room temperature for up to one year; reagent C, 40% trisodium citrate dihydrate stored at room temperature for up to 3 months; color reagent, 3.75 ml reagent A plus 3.34 ml reagent B plus 2.9 ml water, prepared each day. For enzyme assays the enzyme mix and substrate mix were both prepared in 25 mM histidine buffer, pH 8.0, containing 50 mM NaCl and 5 mM MgS0₄, as follows: enzyme mix, 12.5 μg per ml MshC plus 50 mU per ml E. coli pyrophosphatase; substrate mix, 100 μM GlcN-Ins, 200 μM Cys, 200 μM ATP, and 2 mM dithiothreitol.

Preliminary studies were conducted on a Beckman DU 640 spectrophotometer using a 1 cm path length cell containing 0.8 ml of the test solution to which 0.2 ml of color reagent was added to initiate reaction. To quench color development, 50 μl of 40% sodium citrate (reagent C) was added after two minutes with mixing.

In the microtitre plate assay, 80 μl of substrate mix was added to each well of a 96 well microtitre plate (Nunc 269620). Reaction was initiated by addition of 80 μl of enzyme mix and allowed to continue for the desired time when it was halted by addition of 40 μl of the acidic color reagent. Two minutes later further color development was stopped by addition of 10 μl of reagent C. Values of A₆₅₀ were routinely read 20 min after the quenching of color development, but showed little variation (<0.02 absorbance units) up to 60 min after quenching.

Inhibitor screening. Inhibitor dissolved in Spectragrade Me₂SO was added in a volume of 2 μl to 80 μl of substrate mix 2 min prior to addition of 80 μl of enzyme mix. Me₂SO lacking inhibitor (2 μl) was added to 5 wells located on each plate to serve as the positive control defining 100% activity. Negative controls consisting of the complete assay mixture minus GlcN-Ins were included in 3 wells of each plate. Plates were incubated at 23° C. for 60 min to allow reaction to occur prior to addition of the color reagent.

HPLC assay. Enzymatic reactions to determine inhibitor IC50 values were run in 25 mM histidine buffer (pH 8.0, containing 50 mM NaCl and 5 mM MgSO₄) with 0.1 mM ATP, 0.1 mM Cys and 50 μM GlcN-Ins. A 10 μl aliquot of substrate mix was mixed with 1 μl of Me₂SO containing the inhibitor in a 96 well V-bottom plate (Costar). The reaction was initiated 2 min later by addition of 10 μl of enzyme mix and the mixture incubated at 23° C. for 60 min. Where required, Triton X-100 was added to the substrate mix at twice the desired final concentration. Reaction was quenched by addition of 10 μl of 10 mM mBBr in 10% aqueous acetonitrile and incubation in the dark for 15 min at 23° C. The samples were diluted with 70 μl of 10 mM methanesulfonic acid and analyzed by HPLC. HPLC analysis of CySmB-GlcN-Ins was conducted as previously described (Sareen et al., 2002).

Inhibitor studies with Mycobaterium smagmatis. An exponential culture of M. smegmatis mc²155 in Middlebrook 7H9 medium containing 0.05% Tween 80 and 1% glucose was used to inoculate a culture in the same medium but with 0.4% glucose to A₆₀₀=0.05. The inhibitor NTF1836 was added from a stock solution in Me₂SO at 0, 10, 20, 30 and 40 μM to duplicate 40 ml aliquots of the culture in 125 ml flasks to produce a final Me₂SO content of ≦0.04%. The cells were incubated at 37° C. and 250 rpm. At A₆₀₀˜0.25 and subsequent doublings in cell density, a volume of culture was removed sufficient to provide analysis of GlcN-Ins and MSH as described above.

HPLC assays for MshA activity. An HPLC protocol was developed using tetrabutylammonium ion pairing that separates the substrates and products of the MshA catalyzed reaction based upon the number of phosphates. Substrates and products with no phosphates (GlcNAc, GlcNAc-Ins, uridine) are separated from products with one, two or three phosphates as shown in FIG. 16. This assay has sufficient sensitivity for detection of UDPGlcNAc and UDP to follow MshA assays with crude extracts at 260 nm. It is much less sensitive for intermediates and products without absorbance at 260 nm, such as GlcNAc-Ins. To search for intermediates in the production of GlcNAc-Ins this chromatography was used in conjunction with 3H-UDP-GlcNAc [glucosamine-6-3H(N)] and monitoring of the eluent by scintillation counting.

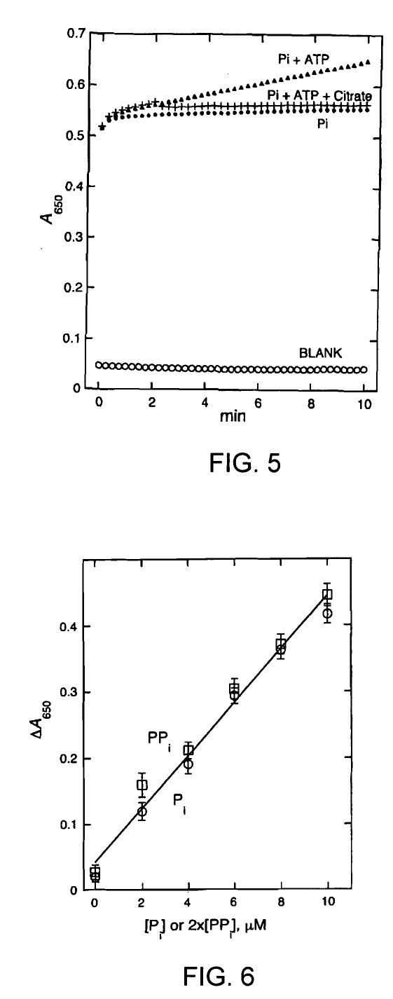

Initial studies were undertaken to establish the optimal conditions for determination of phosphate derived from orthophosphate in the presence of pyrophosphatase. There are many variations on the assay of phosphate via its complex with molybdate, but the most sensitive (Cogan, et al., A robotics-based automated assay for inorganic and organic phosphates, Anal. Biochem. (1999) 271,29-35) involve variations on the original method of Itaya and Ui (Itaya, et al., A new micromethod for the colorimetric determination of inorganic phosphate, Clin. Chim. Acta (1966) 14,361-366) based upon colorlmetric determination of the malachite green complex of phosphomolybdate. The protocol described herein is a modified version of that described by Shatton, et al. (Shatton, et al., A microcolorimetric assay of inorganic pyrophosphatase, Anal. Biochem. (1983) 130, 114-9). One problem with this method is that ATP hydrolysis in the acidic solution employed to produce the colored complex causes the A₆₅₀ value to increase with time, but this can be overcome by quenching the color reaction with citrate (Lanzetta, et al., An improved assay for nanomole amounts of inorganic phosphate, Anal. Biochem. (1979) 100, 95-7). For the MshC catalyzed reaction, 0.1 mM ATP is near the saturation concentration. As shown in FIG. 5 the color development with phosphate alone is complete in less than a minute but in the presence of 0.1 mM ATP the A₆₅₀ value continues to slowly increase with time. The development of the color complex between molybdate and phosphate was quenched with citrate to eliminate the increase in A₆₅₀ due to ATP hydrolysis (FIG. 5). Based upon these results a protocol was established in which the color reaction was allowed to develop for two min and then quenched with citrate.

FIG. 4 shows standard curves for phosphate and pyrophosphate generated in the buffer system with substrates used for enzyme assay. For both phosphate and pyrophosphate the buffer also contained pyrophosphatase (50 mU per ml) and the sample was incubated 1 min at room temperature which allowed full cleavage to phosphate prior to addition of the color reagent. The A₆₅₀ value increases linearly over the range of phosphate concentration shown but exhibits downward curvature at higher concentrations. This problem can be circumvented by addition of polyvinyl alcohol (Van Veldhoven, et al., Inorganic and organic phosphate measurements in the nanomolar range, Anal. Biochem. (1987) 161, 45-8), but this was not necessary for the concentration range (1-10 μM; FIG. 6) covered by the present assay.

In order to apply this assay to measure MshC activity, a number of resources are required. Most of the reagents are commercially available but MshC and GlcN-Ins cannot presently be purchased and must be prepared from cellular sources. The enzyme employed was the native MshC (Rv2 130c) from M tuberculosis expressed in a M smegmatis mshC mutant (strain I64) (Rawat, et al., Mycothioldeficient Mycobacterium smegmatis mutants are hypersensitive to alkylating agents, free radicals and antibiotics, Antimicrob. Agents Chemother. (2002) 46,3348-3355) and purified by conventional means (as provided above). The GlcN-Ins was obtained from MSH using a modification of a published procedure in which MSmB derived from M smegmatis is hydrolyzed by recombinant M tuberculosis mycothiol S-conjugate amidase (Steffek, et al., Characterization of Mycobacterium tuberculosis mycothiol S-conjugate amidase, Biochemistry (2003) 42, 12067-12076) to produce GlcN-Ins and AcCySmB that are readily separated (as provided above).

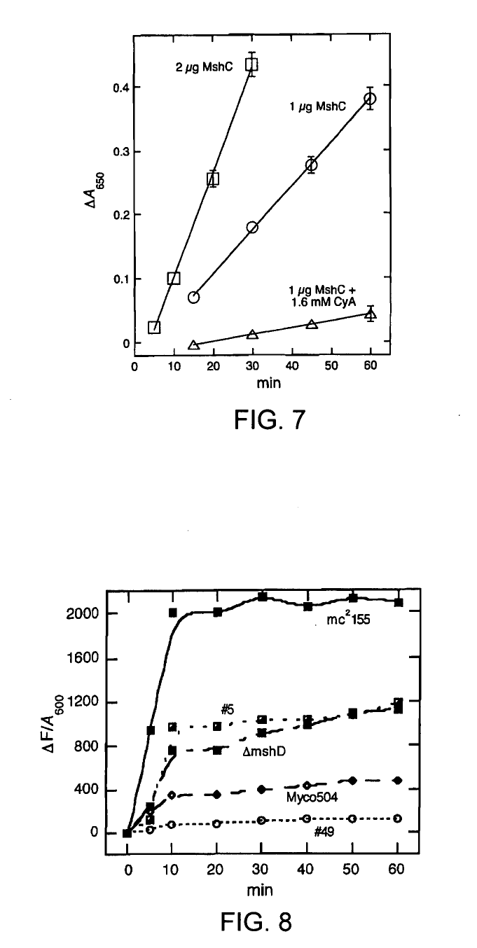

As standard conditions for the assay of MshC final concentrations of 100 μM ATP, 100 μM Cys, and 50 μM GlcN-Ins were employed. FIG. 7 shows results obtained using a 96-well plate format. Reactions were initiated by adding 80 μl of enzyme mix to an 80 μl aliquot of substrate mix. Reactions were quenched at varying time intervals by addition of the acidic color reagent and the color reaction quenched two minutes later by addition of 10 μl concentrated sodium citrate. The A₆₅₀ values increase linearly with enzymatic reaction time over a range corresponding to a maximum of ˜10% conversion of the limiting substrate (FIG. 7). The rate increases with an increase in the concentration of MshC but is markedly depressed by the presence of 1.6 mM cysteamine (FIG. 7). Cysteamine is structurally similar to Cys but lacks the carboxyl group and therefore cannot function as a substrate in the MshC catalyzed reaction. It was found to be a weak inhibitor of the reaction catalyzed by MshC using the HPLC assay. Its marked suppression of the MshC activity in the coupled-enzyme assay (FIG. 7) demonstrates the utility of this assay for screening of potential inhibitors of MshC.

As an initial test of the applicability of this assay for screening of inhibitors, a library of 2024 compounds, purchased from Chemical Diversity, were tested as candidate inhibitors of ATPdependent enzymes. Compounds were dissolved in dimethyl sulfoxide and added to the substrate mix prior to addition of the enzyme mix to produce a final concentration of 100 μM and 2.5% dimethyl sulfoxide. Each plate contained controls with Me₂SO but no inhibitor; a negative control lacking GlcN-Ins yielded the basal A₆₅₀ value that was subtracted fiom all measurements and a positive control including GlcN-Ins was used to define 100% activity.

The majority of the problems in the screening assay derive from inherent properties of the compounds tested. Light scattering due to turbidity or absorbance at 650 nm can produce A₆₅₀ values in excess of the positive control. Certain inhibitors react under the acidic conditions produced by the color reagent to generate colored compounds that have interfering absorbance. Inhibitors of pyrophosphatase can block the conversion of pyrophosphate to phosphate and thereby generate a false positive hit. The concentration of pyrophosphatase used in the assay is many-fold higher than required in order to reduce such false positives but this does not suffice to completely eliminate this factor. However, a secondary HPLC assay to directly measure Cys-GlcN-Ins produced in the MshC catalyzed reaction serves to discriminate between false positives and true inhibitors of MshC initially identified by the screening assay.

Of the 2024 compounds examined at 100 μM, 65 failed to give measurable results in the screening assay owing to insolubility in the final assay mix or to anomalous absorbance by the inhibitor in acid solution. Of the remaining 1959 compounds, 137 (7%) produced inhibition greater than 50%. This high percentage of hits suggested that promiscuous inhibition (McGovern, et al., A common mechanism underlying promiscuous inhibitors from virtual and high-throughput screening, J. Med. Chem. (2002) 45, 1712-22; and McGovern, et al., A specific mechanism of nonspecific inhibition, J. Med. Chem. (2003) 46,4265-72) might be a significant factor. Such promiscuous inhibition is thought to involve aggregates of the inhibitor that associate with proteins and disrupt enzyme activity. Such aggregation can be prevented by detergents such as Triton X-100 (McGovern, et al., 2003; and Ryan, et al., Effect of detergent on “promiscuous” inhibitors, J. Med. Chem. (2003) 46, 3448-51). It was established that Triton X-100 up to 0.05% in the assay produces only a minor increase (≦15%) in MshC activity in the HPLC assay. However, Triton X-100 above 0.01% produced ˜50% increase in the A₆₅₀ values for the screening assay in 96-well plates and the Triton X-100 concentration was therefore limited to 0.005% for the screening assay. The 137 compounds that gave >50% inhibition at 100 μM concentration were further examined at 30 μM concentration in the presence of 0.005% Triton X-100 using the HPLC assay for Cys-GlcN-Ins production and only 7 showed evidence of minor inhibition (>22% inhibition). This suggested that many of the compounds were pyrophosphatase inhibitors. A counterscreen was tested at 30 μM inhibitor and 5 μM pyrophosphate with 0.005% Triton X-100 in the absence of MshC and GlcN-Ins as in FIG. 6. The results showed that about half of the compounds produced inhibition >22%. Thus, use of a pyrophosphatase counterscreen to reduce false positive hits could have reduced the false positive hit rate by half to ˜3%. The secondary HPLC screen further reduces it to ˜0.4% (7/1959).

The 7 hits confirmed by HPLC were examined further. The concentration dependence was tested at 50, 100 and 200 μM in 0.005% Triton X-100 and only one of the seven compounds exhibited clear concentration-dependent inhibition. This compound, designated NTF 1836, gave a linear reciprical plot of velocity versus inhibitor concentration with an IC50˜100 μM.

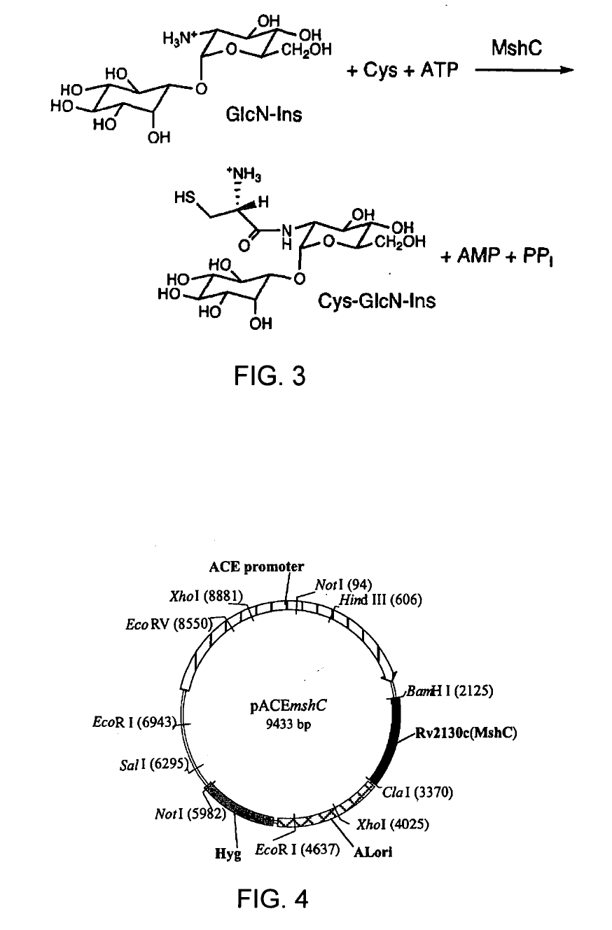

When NTF 1836 was tested on M. smegmatis mc²155 at 40 μM, growth was completely inhibited after 8 h. In 30 μM NTF1836, M. smegmatis grew at half the normal rate and HPLC analysis showed that after 12 h the GlcN-Ins content was twice the normal level and the MSH content was 40% of the level in the absence of inhibitor. This is the pattern predicted for a MshC inhibited strain (FIG. 3). In the extreme case of a mshC genetic knockout in M. smegmatis, MSH content was 0-2% of normal and GlcN-Ins accumulated to a level 20-25-fold higher than normal. In the present case the reduction in MSH is suggestive of MshC inhibition, but not conclusive since non-viable cells might have a low MSH content. However, the accumulation of GlcN-Ins is difficult to explain by cell death and is more compelling evidence of MshC inhibition. Blockiung MSH production does not prevent growth of M. smegmatis (Newton, et al., Characterization of a Mycobacterium smegmatis mutant defective in 1-D-myo-inosityl-2-amino-2-deoxy-α-D-glucopyranaonside and mycothiol biosynthesis, Biochem. Biophys. Res. Commun. (1999) 255, 239-24) so inhibition of MshC cannot explain the growth inhibition. It is probable that this inhibitor has targets other than MshC and a possible candidate is the CysS cysteinyl-tRNA synthetase which is homologous to MshC.

Although the coupled enzyme assay is useful for inhibitor screening with purified MshC, its application for the determination of MshC activity in cellular extracts and other crude preparations has limitations owing to the possible presence of ATPase activity. This can be measured in a control experiment in which GlcN-Ins is excluded from the substrate mix. When the assay was applied to a 20-45% saturated ammonium sulfate fraction from a crude extract of M. smegmatis strain mc²155 there was no significant difference between the control and complete assay results. For this native M. smegmatis strain the ATPase activity substantially exceeds the MshC activity. However, when the corresponding fraction from the MshC overexpressing strain (I64::pACEmshC) was assayed by the coupled enzyme assay, the control activity was only half the complete assay value, and after correcting for the control activity the net activity corresponded with the result obtained from direct assay of Cys-GlcN-Ins production by HPLC. Thus, the coupled enzyme assay is useful only when the MshC activity is quite high or the ATPase activity has been diminished by protein fractionation.

Thus, the coupled-enzyme assay described here provides a method useful in screening for drugs directed against MshC, an essential enzyme for growth of M. tuberculosis. Inhibitors of the pyrophosphatase coupling enzyme can be identified in a counterscreen assay, and a secondary HPLC assay is available to validate true positive hits. Incorporation of Triton X-100 is required in all assays to avoid promiscuous inhibition. Application of the coupled-enzyme assay to detect MshC activity in complex mixtures is limited to those in which MshC activity exceeds ATPase activity.

### Example 2

### Characterization of M.tuberculosis MshC

Middlebrook 7H9 was purchased from Difco Laboratories, and glucose and Tween 80 were from Fisher. MSH was isolated from M. smegmatis as described (Unson, et al, (1998) J. Immunol. Meth. 214, 29-39.) and the monobromobimane (mBBr, Molecular Probes) derivative (MSmB) was prepared and purified by the method of Newton, et al. (1995) Methods Enzymol. 251, 148-166. GlcN-Ins was prepared by the quantitative hydrolysis of MSmB by purified M. smegmatis mycothiol S-conjugate amidase as previously described (Newton, et al. (2000) Biochemistry 35, 10739-10746.). CySmB-GlcN-Ins was purified by preparative HPLC, after acid hydrolysis of MSmB, as described (Anderberg, et al. (1998) J. Biol. Chem. 273, 30391-30397.).

Analysis of MSH and the precursors GlcN-Ins, GlcNAc-Ins. Cells were extracted and derivatized with mBBr for thiol analysis as previously described (Koledin, et al. (2002) Arch. Microbiol. 178, 331-337.). The mycothiol precursors, GlcN-Ins and GlcNAc-Ins, were measured by the method of Buchmeier, et al. (Buchmeier, et al. (2003) Mol. Microbiol. 47, 1723-1732.).

MshC ligase assay. The standard protocol for determination of MshC activity during enzyme purification was that described by Sareen, et al., (2002) Biochemistry 41, 6885-6890. For kinetic studies with the natural substrates (ATP, L-Cys and GlcN-Ins) the concentration of one substrate was varied, keeping the other two constant in the presence of ˜100 ng of purified M. tuberculosis MshC. Protease inhibitors were omitted in the kinetic studies. Alternative substrates to Cys were analyzed at 80, 200, 800 and 1600 μM with 50 μM GlcN-Ins and 1 mM each of ATP, MgCl₂ and DTT in 50 mM HEPES pH 7.5 containing ˜100 ng of purified MshC. The reaction mixtures were incubated at 37 ° C. and sampled at 4 and 40 min. For the substrates containing the thiol group, the assay mixture was derivatized with mBBr by the standard derivatization procedure and assayed for the corresponding thiol product (i.e. MSH from AcCys). For the non-thiol substrates (e.g. L-alanine) the MshC ligase activity was determined by assay of AMP production as described below.

Cys-tRNA synthetase assay. The cys-tRNA synthase activity of MshC was examined using a modification of the methods of Schrier and Schimmel (Schreier, et al. (1972) Biochemistry 11, 1582-9.). Purified E. coli cys-tRNA synthetase was used as a positive control for this reaction and was a generous gift from Kirk Beebe and Paul Schimmel of The Scripps Research Insitiute, La Jolla, Calif. Previous studies indicate that mycobacterial tRNA synthetases will charge E. coli tRNAs (Kim, et al. (1998) FEBS Lett 427, 259-62.). Measurement of the formation of tRNA^{cys} was determined by the separation of free ¹⁴C-cysteine from ¹⁴C-tRNA^{cys} by the filtration of TCA precipitates (Schrier and Schimmel, 1972, supra). Acid precipitated counts are assumed to be ¹⁴C-tRNA^{cys} and control reactions without tRNAs were used to estimate background filter counts.

Whatman GF/C 25 mm glass fiber filters were prewashed in 7% TCA and dried prior to use. E. coli cys-tRNA synthetase (15 μg) or purified M. tuberculosis MshC (34 μg) were assayed in 2 mM ATP, 4 mM MgCl₂, 20 mM DTT, 20 mM KCl, 0.1 mg/ml bovine serum albumin (Sigma), 10 mg/ml E. coli tRNAs (Boehringer Mannheim), 60 μM ¹⁴C-cysteine (18 μCi/μmole, Perkin Elmer), and 50 mM HEPES pH 7.5, final concentrations in an assay volume of 100 μl. Reactions were incubated at 23° C. and 18 μl aliquots were removed from the reaction at 1, 3, 6, 9, and 15 min and mixed with 1 ml of aqueous 7% TCA. These samples were incubated for 10 min at 23 ° C. and vacuum filtered. The sample filters were washed with 1 ml 7% aqueous TCA followed by 5 ml 95% ethanol and dried in a vacuum oven at 50 ° C. Dried filters were counted in 9 ml Econo-Safe scintillation cocktail (Research Products International) in a Beckman model LS1701 scintillation counter.

AMP assay. The formation of AMP was assayed by HPLC with some changes in the method described by Beuerle, et al (2002). The ligase reaction mixture (100 μL) was terminated by the addition of NEM to 2 mM followed by 2 μL of 5 N methanesulphonic acid; it was immediately frozen in dry ice. Analysis for AMP and ATP analysis conducted by HPLC on a Beckman Ultrasphere IP (250×4.6 mm) analytical column fitted with Brownley OD-GU 5 μC-18 cartridge using a flow rate of 1 mL/min and the following linear gradient: 0 min, 0.1% B (77 mM KH₂PO₄, 2.2 mM tetrabutylammonium hydroxide, 38.5% methanol, pH 5.5); 40 min, 100% B; 42 min, 100% A (10 mM KH₂PO₄, 10 mM tetrabutylammonium hydroxide, 0.25% methanol, pH 7.0); 50 min, 100% A (reinjection). The nucleotides were detected at 260 nm on a Waters 486 UV detector.

Inhibition studies. To test for inhibition of MshC ligase activity, ˜100-150 ng of the purified enzyme was incubated with different concentrations of inhibitor in 50 mM HEPES pH 7.5, for 30 min. at room temperature followed by the sequential addition of 1 mM each of DTT, ATP and MgCl₂, 70 μM L-Cys, and 50 μM GlcN-Ins in a final volume of 30 μL. Aliquots (12.5 μL) were withdrawn at 2 min. and 4 min for derivatization with mBBr and HPLC analysis as described earlier (Sareen, et al., 2002, supra.). For MSH and Cys-GlcN-Ins inhibition analysis, the second substrate GlcN-Ins was used at a concentration near the Kₘ value (300 μM) found in this study. The protocol was modified for the product (Cys-GlcN-Ins) inhibition studies to allow measurement of initial rate in the presence of ˜5 μM added Cys-GlcN-Ins. The enzyme level was reduced 10-20-fold to produce a rate capable of producing a measurable increase in Cys-GlcN-Ins.

Metal chelation by phenanthrolines. Stock solutions of 1,10-phenanthroline (Kodak) and 1,7-phenanthroline (Aldrich) were prepared in dimethylsulphoxide. Purified amidase (90 ng) in 42 μL of assay buffer was incubated with 0.1-5 mM (n=4), of phenanthrolines at room temp. for 10 min. The ligase reaction was initiated by the sequential addition of reaction components i.e. 1 mM each of ATP, MgCl₂ and DTT with 1 mM L-Cys and 600 μM GlcN-Ins. After 10 min. of incubation at 37° C., the reaction was stopped and derivatized by the addition of 8 mM mBBr in acetonitrile, and acidified by 10 mM methanesulfonic acid followed by HPLC analysis as described earlier (Id.).

Zn supplementation. Purified MshC (131 ng) was incubated with 2, 10, 50 or 100 μM zinc chloride in 25 μL of the assay buffer at room temp or at 37° C. The ligase reaction was initiated by the sequential addition of 1 mM each of ATP, MgCl₂ and DTT, 100 μM L-Cys and 50 μM GlcN-Ins. At 2 and 4 min 12.5 μL aliquots were withdrawn and analyzed for thiol content as described above.

Cloning of mshC (cysS2) in pACE. mshC/Rv2130c was earlier cloned in pRSETA into BamHI/HindIII sites under T7 promoter in E. coli BL21 DE3 pLysS (Sareen et al., 2002, supra). While there was activity in the soluble fraction, the bulk of the protein was found to accumulate in the form of insoluble inclusion bodies. Attempts to solubilize and reactivate the protein proved unsuccessful. Cloning of the native MshC protein and expression in M. smegmatis as a more suitable host was therefore examined.

The expression was then tried in M. smegmatis under the acetamidase promoter of pACE (DeSmet et al., (1999) Microbiol. 145, 3177-3184) and pALACE (Koledin et al., 2002, supra) vectors, at the restriction sites, BamHI and ClaI. The mshC gene in pRSETA was restriction digested with BamHI and Hind III, subcloned in the vector pSODIT-2 at the two respective sites. The gene was then digested from this vector with BamHI and ClaI and was further subcloned at the respective sites in pACE. The M. tuberculosis mshC (Rv2130c) was cloned into pACE, a shuttle plasmid for E. coli and mycobacteria, having a cloning site downstream of an inducible M. smegmatis acetamidase promoter (De Smet, 1999, supra.) to produce pACE::mshC (FIG. 4). The pACE::MshC was used to electrotransform the M. smegmatis I64 mutant, which is deficient in MshC activity (Rawat, et al. (2002) Antimicrob. Agents Chemother. 46, 3348-3355.). M. smegmatis mc²155 contains native MshC protein, which is translated in more than one form and would contaminate the recombinant M. tuberculosis MshC protein. Thus, a mycothiol mutant, I64, deficient in MshC was used as a host for expression of M. tuberculosis mshC. Strain I64 is deficient in MshC ligase activity due to a Leu205Pro amino acid substitution resulting from a single point chemical mutation (Rawat, et al., 2002, supra) and produces much reduced levels of mycothiol (Table 1).

The electrocompetent cells of mutant I64 were made by repeated washing of the cells cultured to exponential phase (A₆₀₀=0.5), with sterile 10% glycerol. After electroporation, the cells were supplemented with one mL of 7H9+1% Glucose and shaken at 37° C. for 4 hours before plating onto 7H9+1% glucose plates, supplemented with 75 μg/mL hygromycin.

Growth of recombinant MshC culture and pACEMshC expression. M. smegmatis MshC mutant I64 complemented with M. tuberculosis mshC (Rv2130c), hereafter denoted I64::pACEmshC, was grown in 7H9 medium supplemented with 0.05% Tween 80, 10% OADC (BBL) and hygromycin (75 μg/ml) at 37° C. and 250 rpm. The culture was propagated on a large scale in the same media but with 1% glucose instead of 10% OADC. The induction was initiated at A₆₀₀=0.3 with acetamide by centrifuging the cells at 8000 g for 15 min and resuspending them in a new media without glucose and instead, 0.4% acetamide as the carbon source, while maintaining the antibiotic selection pressure. After 28 h of cultivation at 37° C. and 250 rpm, the bacterial cells were collected by centrifugation at 8000 g for 15 min. The cell pellets, about 2.3 g/liter, were stored at −70° C. until further use.

Overexpression of the MshC ligase in pACEmshC transformed M. smegmatis strain I64 with acetamide induction yielded protein in the soluble fraction of the cell-free extract. Following induction of pACEmshC by 0.4% acetamide in the mutant strain I64, the MSH content was measured and found to be complemented to a level 150% that of the wild type strain mc²155 (Table 1). The MSH biosynthesis intermediates, GlcN-Ins and GlcNAc-Ins, were found to accumulate in mutant I64 to a level ˜20-fold higher than that of the wild-type strain. Upon MshC induction in the complemented I64strain the levels of both MSH precursors dropped to values typical of the wild-type strain and the MSH content increased 150-fold to a value above the wild-type level (Table 1).

TABLE 1
Mycothiol and precursor levels in M. smegmatis
parent and mutant strains

| | cellular content | | |
|---|---|---|---|
| | (μmol/(g residual dry weight)) | | |
| M. smegmatis strain | GlcNAc-Ins | GlcN-Ins | MSH |
| mc²155 (parent) | ≦0.2 | 1.0 ± 0.2 | 10 |
| mutant I64 | 4 ± 2 | 19 ± 1 | 0.1 |
| mutant I64::pACEmshC | <0.05 | 0.4 ± 0.1 | 15 ± 1 |

Purification of recombinant ligase. All operations were carried out at 4° C. in the presence of 3 mM 2-mercaptoethanol and 5 mM MgCl₂ unless stated otherwise. Twenty grams of I64::pACEmshC cells (wet wt.) from 9 liter broth was suspended in 80 ml of 50 mM HEPES buffer (25% cell suspension), pH 7.5 in the presence of 35 μM of the protease inhibitors TPCK and TLCK. The cells were disrupted by ultrasonication (Branson Sonifier 200) in an ice bath. The cell debris was removed by centrifugation at 100,000 g for 1 h at 4° C. The supernatant obtained was used as the source of the enzyme. The cell free extract thus obtained was subjected to 20% ammonium sulfate precipitation in ice, for 2 h followed by centrifugation at 28,000 g for 30 min. The supernatant was further subjected to 20%-45% ammonium sulfate precipitation in ice for an overnight and the precipitated proteins were pelleted by centrifugation at 28,000 g for 30 min. The protein pellet (4.8 g) was resusupended in 48 ml of the 50 mM HEPES, pH 7.5 containing 35 μM of TPCK and TLCK and was desalted by passing it through Sephadex G-25 column.

The 150 ml material obtained from G-25 column was applied on DEAE 650-M column (5.2×10.6, 225 ml) preequilibrated with 50 mM HEPES, pH 7.5. The enzyme was eluted at 0.2 M NaCl by running a linear gradient of 0-0.5 M NaCl in 15 column volumes of the buffer at 300 ml/h. The fractions containing the enzyme activity were combined (235 ml) and were diluted to twice the volume (480 ml) with Milli-Q water to lower the salt concentration.

The diluted solution was applied to a Bio-gel HTP column (2.6×11.3, 60 ml) at 120 ml/h, which was pre-equilibrated with 10 mM potassium phosphate buffer, pH 6.8 and 100 mM NaCl. The bound proteins were eluted with a linear gradient of 10 mM to 100 mM phosphate concentration and 100 to 0 mM NaCl concentration in 15 column volumes. The active fractions were collected (76 ml) and fractions 29, 31, 33, 34, 35, 36, 37 were analyzed for purity on 12.5% SDS-PAGE. There were few impurities left, so fractions 29-37 were pooled, precipitated with 80% ammonium sulfate, and taken up in 50 mM HEPES buffer pH 7.5 for gel filtration chromatography on Sephacryl-200 column (247 ml) at 10 ml/h in 50 mM HEPES, pH 7.5 and 150 mM NaCl. (Table 2) Fractions 49-54 were analyzed on SDS-PAGE before pooling. The pooled enzyme was concentrated in 10 kD membrane filters (Sigma) and stored in 50% glycerol at −70° C. in 30 μL aliquots for the detailed characterization studies.

Ligase activity was found to be soluble in the cytoplasmic fraction on sonication of the cells and the level of activity was found to be ˜400-fold greater than in M. smegmatis mc²155 (Sareen, et al., 2002, supra.) the parent strain of mutant I64. The recombinant protein eluted as a single peak with apparent Mᵣ=34 kD on the S-200 column (actual Mr 45,591). Thus, the M. tuberculosis MshC protein exists as a monomer in its native form. This contrasts with its ortholog from M. smegmatis, which forms dimers and tetramers in the native state (Sareen, et al., 2002, supra.).

TABLE 2
Purification of M. tuberculosis Cys: GlcN-Ins ligase (MshC)

| | | | specific | | |
|---|---|---|---|---|---|
| | | | activity | | |
| | protein^{a} | total activity | (nmol | yield | Purif. |
| Step | (mg) | (nmol min⁻¹) | min⁻¹ mg⁻¹) | (%) | factor |
| Crude extract | 1670 | 14600 | 8.7 | (100) | (1) |
| 20-45% SAS | 540 | 14000 | 26.0 | 96.0 | 3.0 |
| DEAE ion | 94 | 2590 | 27.5 | 17.7 | 3.7 |
| exchange | | | | | |
| Hydroxyl | 10 | 1076 | 108 | 7.4 | 12.4 |
| apatite | | | | | |
| S-200 gel | 0.6 | 93 | 155 | 0.64 | 17.8 |
| filtration | | | | | |

^{a}Protein concentration based upon A₂₈₀ value, where 1 AU = 0.58 mg/ml for purified MshC.

Metal analysis. The purified enzyme was analyzed by inductively coupled plasma-atomic emission spectroscopy (ICP) at the San Diego Gas & Electric Environmental Analysis laboratory for 26 metal ions; Al, Sb, As, Ba, Be, B, Cd, Ca, Cr, Co, Cu, Fe, Pb, Mg, Mn, Mo, Ni, K, Se, Si, Na, Sr, Th, Ti, V and Zn. The enzyme sample was diluted to 1 mg/ml and buffer was also submitted for background metal analysis.

The ICP showed that there is 0.7 mol of Zinc ion/mol of enzyme. Attempts to inactivate the enzyme by chelating the metal present with 1,10-phenanthroline produced no significant loss of activity and supplementation of the enzyme with Zn²⁺ led to a loss in activity. Thus, if Zn²⁺ is required for activity, it must be quite tightly bound and not easily removed or complexed by 1,10-phenanthroline. In addition there must be residues capable of binding Zn²⁺ in a fashion which distorts or blocks the active site in a fashion which interferes with enzyme activity.

Testing of MshC for Cys-tRNA ligase activity. The initial reaction of cysteinyl-tRNA synthetase and MshC is the formation of enzyme bound AMP-cys. Since E. coli cys-tRNA synthetase and M. tuberculosis MshC (cysS2) are homologs, they have been used to focus on the second reaction, the tRNA charging reaction of cys-tRNA synthetase and MshC. It was found that the reactions were complete in 1 min at room temperature and gave stable filter counts after that time. The rates for the formation of tRNA^{cys} were 11±2.5 (n=5) and 0±0.2 (n=4) nmole/min/mg protein for E. coli cys-tRNA synthetase and M. tuberculosis MshC, respectively. Thus M. tuberculosis MshC, annotated as cysS2 or cysteinyl-tRNA synthetase 2, will not charge E. coli tRNAs in this assay. The foregoing shows that the activity of MshC is the ATP dependent formation of Cys-GlcN-Ins, an intermediate in the mycothiol biosynthesis pathway, and not the synthesis of tRNA^{cys}.

Stoichiometry of the reaction catalyzed by MshC. Purified MshC was utilized to establish the stoichiometry of the reaction catalyzed. It had been initially assumed that the ATP-dependent production of Cys-GlcN-Ins yielded ADP as a coproduct by analogy with the enzymology of γ-glutamylcysteine biosynthesis, the intermediate precursor of glutathione (Newton, et al. (2000) J. Bacteriol. 182, 6958-6963; Bornemann, et al. (1997) Biochem. J. 325, 623-9.). However, when MshC was identified as a homolog of cysteinyl-tRNA synthetase (CysS), where the overall reaction produces AMP plus pyrophosphate, it appeared likely that the product of the MshC catalyzed reaction was also AMP. To verify this the levels of Cys, ATP, AMP and Cys-GlcN-Ins were monitored over 60 min in a reaction initiated with 100 μM Cys, 100 μM GlcN-Ins, and 200 μM ATP. After 60 min the reaction was 30% complete and for each equivalent of Cys utilized 0.82 equivalents of Cys-GlcN-Ins was produced, accompanied by the utilization of 1.19 equivalents of ATP and the production of 0.95 equivalents of AMP. This establishes the reaction stoichiometry as indicated in FIG. 3.

Enzyme kinetics and substrate specificity. The factors influencing the enzymatic assay of MshC activity at 37° C. were explored in 50 mM HEPES buffer, pH 7.5 containing 1 mM DTT. With 100 μM GlcN-Ins, 0.5 mM Cys, and 0.5 mM ATP as substrates, the reaction rate increased sharply with Mg²⁺ concentration up to 100 μM, then leveled and became constant from 1-5 mM Mg²⁺. Under the same conditions but with 5 mM Mg²⁺ and varying the ATP concentration, the rate increased up to 100 μM ATP, remained constant to 1 mM ATP, and then declined 2.2-fold at 5 mM ATP. Based upon these results 1 mM each of Mg²⁺ and ATP were selected as standard assay concentrations. The apparent Kₘ values were estimated from Eadie-Hofstee plots, which were linear over the indicated range of concentration. With 1 mM each of ATP and GlcN-Ins, the apparent Kₘ for L-Cys (5 μM to 1.6 mM, n=10) was determined to be 85±20 μM and the apparent Vₘₐₓ was 1450±200 nmol min⁻¹ mg⁻¹. For 1 mM ATP and 0.5 mM Cys, the apparent Kₘ for GlcN-Ins (10 μM to 3 mM, n=6) was determined to be 280±43 μM and the apparent Vₘₐₓ was 1160±120 nmol min⁻¹ mg⁻¹. The apparent Kₘ for GlcN-Ins of 280±43 μM is about 4-fold higher than the value reported for the enzyme purified from M. smegmatis.

Several thiols related to cysteine were tested as alternative substrates to Cys, each examined at concentrations ranging from 80 μM to 1.6 mM. The results obtained at the highest concentration are given in Table 3. The enantiomer, D-Cys, was a poor substrate. Neither β-mercaptopropionic acid nor cysteamine, derived from Cys by removal of the amino and carboxyl groups, respectively, produced evidence of reaction. Nor was significant activity detected with AcCys, L-homocysteine, L-serine, or L-alanine. Thus, the enzyme is highly specific for Cys. The specificity is less stringent for GlcN-Ins, with GlcN having ˜1% the activity of GlcN-Ins increasing linearly over the range of concentration studied.

Inhibition of MshC. Various compounds were tested over the concentration range 80-1600 μM as inhibitors of the ligase activity measured with 70 μM L-Cys and 50 μM GlcN-Ins. Results for the highest level tested are shown in Table 3. Only very minor inhibition was produced by the amino acids D-Cys, L-homocysteine, L-α-aminobutyric acid, L-serine, and L-alanine. β-mercaptopropionic acid (deaminated Cys) and N-acetylcysteine also produced minor inhibition. Of all compounds tested the best inhibitor was cysteamine (decarboxylated Cys) which produced a 3-fold reduction in rate at 1.6 mM. Mycothiol also produced minimal inhibition at concentrations of 1 and 5 mM, representing physiologic levels.

TABLE 3
Substrate specificity and inhibition of MshC

| | relative | inhibition | |
|---|---|---|---|
| compound | spec. act.^{a} | [inhibitor] (μM) | % inhibition^{b} |
| Cys (70 μM) | (100)^{c} | — | — |
| D-Cys | ≦0.7 | 1600 | 15 ± 8 |
| β-mercaptopropionate | ≦0.004 | 1600 | −20 ± 20 |
| cysteamine | ≦0.16 | 1600 | 65 ± 10 |
| AcCys | ≦0.24 | 1600 | 20 ± 6 |
| L-homocysteine | ≦0.2 | 1600 | 0 ± 5 |
| L-serine | ≦0.008 | 5000 | 15 ± 8 |
| L-alanine | ≦0.016 | 5000 | 4 ± 8 |
| D-GlcN (1.6 mM)^{d} | 1.2 | — | — |
| MSH | — | 1000 | 4 ± 3 |
| MSH | — | 5000 | 7 ± 5 |

^{a}Ligase activity determined at 37° C. with 50 μM GlcN-Ins and 1.6 mM test substrate, unless otherwise noted. Relative specific activity calculated from maximal peak intensity at highest test concentration and assuming linear dependence upon concentration.
^{b}Inhibition determined with 50 μM GlcN-Ins and 70 μM Cys under standard assay conditions.
^{c}Specific activity 108 nmol min⁻¹ mg⁻¹.
^{d}In place of GlcN-Ins with 100 μM Cys.

The MshC ligase was also tested for feedback inhibition by Cys-GlcN-Ins. The intracellular level of Cys-GlcN-Ins was found to be 5-10 μM in M. tuberculosis, when analyzed at different growth time points (Buchmeier, Newton, Koledin and Fahey, unpublished). So, it was logical to analyze Cys-GlcN-Ins in the concentration range of 1-10 μM with levels of the Cys and GlcN-Ins substrates near their Kₘ values, 70 and 300 μM, respectively. The apparent Kₘ value for Cys of 70±15 μM found here is nearly double the value found earlier for the M. smegmatis enzyme. Cys-GlcN-Ins produced <1.0 and 3.6% inhibition when tested at 0.7 μM and 6.6 μM, respectively, which shows that there is no significant feedback inhibition at physiological levels of the reaction product.

### Example 3

### Essentiality of Mycothiol in M. tuberculosis

The use of conditional null mutants to establish essentiality in M. tuberculosis has not yet been accomplished so the present example employed the general approach used by Parish and Stoker (Parish, et al. (2000), J. Bacteriol. 182:5715-20) to test the essentiality of the glnE. A second copy of the mshC gene was introduced into wild type M. tuberculosis using an integrative vector pCV125 (kindly provided by MedImmune) which was modified to contain the spectinomycin/streptomycin (Sp/Sm) cassette from pKRP13. This vector containing the mshC gene has been constructed and tested on M. smegmatis strain I64, a chemical mutant defective in mshC and MSH production (Rawat, 2002, supra.). It was shown to be effective in restoring MSH production in M. smegmatis I64. pCV125 integrates into the att site in the M. tuberculosis chromosome and will stably introduce a second copy of the mshC gene into a second location of the chromosome. The mshC ORF plus its ribosomal binding site (71 bp upstream of the ATG start codon) was amplified by PCR using genomic M. tuberculosis (Erdman) DNA. The forward primer 5′-TCCCCCGGGACGCGTGGCGCTGAT-3′ (SEQ ID NO: 1), contains a SmaI restriction site, and the reverse primer 5′-GGACTAGTCTACAGGTCCACCCCGAGCAG-3′ (SEQ ID NO: 2), contains a SpeI restriction site which was used for directional cloning. The PCR fragment was ligated with pCR 2.1 (Invitrogen) using T4 DNA ligase and used to transform TOP 10F′ (Invitrogen) E. coli. After selection on agar plates (LB, ampicillin 100 μg/ml) and growth in broth, plasmid DNA was analyzed by restriction analysis and sequencing. The SmaI/SpeI fragment containing the mshC gene from this plasmid was cloned between the SmaI and SpeI sites within the aph gene in pCV125. This resulted in a vector containing a copy of the mshC gene that is transcribed from the aph promoter. Vector DNA was introduced into wild type M. tuberculosis by electroporation with selection on 7H11 plates containing streptomycin. As a control, pCV125 with no extra DNA was introduced into other aliquots of M. tuberculosis. Streptomycin resistant colonies were grown up, chromosomal DNA was extracted, and the presence of 2 copies of the mshC gene were confirmed by Southern hybridization. NcoI digests were initially used because this enzyme cuts outside of the mshC gene and will allow for easy identification of differences in flanking sequences between the native copy of mshC and the introduced copy. SacI digests were also used to analyze the original and the introduced copies of mshC within the genomic DNA of transformants.

After the presence of the second copy of the mshC gene was confirmed using Southern hybridization, 2X-mshc was infected with the specialized transducing phage containing the mshC knockout construct. The specialized transducing phage is a variant of a mycobacteriophage described by Bardarov et al. (Bardarov, et al. (2002), supra; Bardarov, et al. (1997) Proc. Natl. Acad. Sci. 94:10961-6.) and was a gift from J. Cox. The specialized transducing phage containing the mshC knockout DNA was constructed by amplifying ˜500 bp of the upstream fragment (protein N-terminal region of mshC) comprising 102 bp of the mshC gene and 370 bp of downstream sequence, and ˜500 bp of a middle fragment (residues 195-708) of the mshC gene. This results in a deletion of the residues encoding the active region of the protein. Each primer set incorporated suitable endonuclease sites to allow subcloning of the PCR product into plasmid pJSC284 such that a hygromycin resistance cassette was inserted within the mshC ORF. After verifying the fragment incorporation, the plasmid was digested with PacI, treated with alkaline phosphatase to prevent plasmid rejoining during subsequent ligation and ligated into the PacI site of the specialized transducing phage phAE87. DNA was packaged into λ phage using Gigapack III Gold packaging extract (Stratagene) and this was used to infect HB101 E. coli grown on maltose to promote phage uptake. Colonies were selected on hygromycin. Cosmid DNA was extracted from the hygr colonies and was used to transform M. smegmatis mc²155. The transformation plates were incubated at 30° C. until plaques appeared (2-3 days). Plaques were picked and a high titer phage stock was prepared from M. smegmatis mc²155.

For infection of M. tuberculosis, 10 ml of bacteria was washed with MP buffer and resuspended in 1 ml MP buffer at 39° C. (Buchmeier et al. (2000) Molec. Microbiol. 35:1375-1382.). Phage was added at a multiplicity of infection of 10 and the mixture was incubated at 39° C. for 4 hr to allow for phage infection. The bacteria were spun down, resuspended in 500 ml MP buffer and plated on 7H11 plates containing hygromycin (50 mg/ml). Hygromycin resistant colonies appeared in 3 to 5 weeks. Individual colonies were grown up for analysis by Southern hybridization (to verify the presence of the mshC knockout) and for measurement of MSH content. The allelic exchange substrate should recombine preferentially with the native copy of the mshC gene and not with the introduced copy since the flanking sequences of the introduced mshC gene differ from the flanking sequences of mshC in the phage mutagenesis construct. The corA knockout phage construct was included as a positive control for the transduction procedures.

In initial experiments with M. tuberculosis with only the original copy of mshC, the phAEΔmshC phasmid produced hygromycin resistant transformants that contained parental mycothiol levels. This experiment was repeated several times with a total of 67 hygromycin clones analyzed by Southern blot. All clones were found to have the original copy of mshC intact. Representative clones such as clone 49 (Table 4) were examined for mycothiol content and found to have the parental levels of mycothiol. Thus, no authentic mshC mutants were detected in the 67 clones examined from transformation of the parental strain of M. tuberculosis Erdman.

Initially the integrative plasmid delivering the second copy of mshC was tested on a chemical mutant of mshC in M. smegmatis (strain I64) which produces about 1% of the parental level of mycothiol (Rawat, et al., 2002, supra.). Incorporation of the M. tuberculosis mshC gene resulted in the production of 150% of the parental level of mycothiol in this mycothiol mutant (results not shown). This same plasmid was used to transform M. tuberculosis Erdman to make the merodiploid host 2× mshc identified as strain 1682 in a Southern blot. There is a single SacI site in mshC which generates 2 bands in the original copy observed in the M. tuberculosis Erdman (Erd) strain as well as in the second copy as observed in 1682, the 2× mshC host strain in another Southern blot. The mycothiol content 2× mshC strain is nearly twice the parental level (Table 4). One of the substrates of MshC, GlcN-Ins, is reduced by 64% by the presence of the second copy of mshC in the 2× mshC strain 1682 (Table 4). This is consistent with ligase substrate depletion due to a higher level of cellular MshC activity. The Southern blots taken together with the mycothiol analyses verify the presence of a functional second copy of mshC in the 2× mshC host strain.

TABLE 4
Mycothiol and precursor levels in M. tuberculosis Erdman
mshC knockout strains^{a}.

| | | | |
|---|---|---|---|
| OD₆₀₀ | GlcNAc-Ins | GlcN-Ins | MSH |

nmoles/10⁹ Cells

Host and Control strains

| | | | | |
|---|---|---|---|---|
| M. tuberculosis | 0.50 | 1.7 ± 0.7 | 8.9 ± 0.2 | 13.7 ± 0.2 |
| Erdman (parent) | | | | |
| Strain 3 | 0.41 | ≦0.7 | 7.3 ± 0.1 | 12.1 ± 1.3 |
| 1682 Erdman (+2nd | 0.85 | 0.9 ± 0.1 | 3.2 ± 0.1 | 26.2 ± 1.2 |
| copy MshC) | | | | |
| Strain 49 | 0.47 | 1.7 ± 0 | 9.2 ± 0.1 | 21.5 ± 0.5 |

mshC knockout strains in (2x mshC) M. tuberculosis Erdman 1682

| | | | | |
|---|---|---|---|---|
| Strain 14 | 0.36 | ≦1.4 | 14.1 ± 0.2 | 12 ± 0.3 |
| Strain 16 | 0.37 | ≦0.6 | 5.7 ± 0.3 | 14.6 ± 0.1 |
| Strain 18 | 0.43 | ≦1.2 | 12 ± 1 | 10.2 ± 0.1 |
| Strain 157 | 0.58 | 1.5 ± 0.7 | 12 ± 1.4 | 10.2 ± 0.1 |
| Strain 158 | 0.52 | 1.7 ± 0.4 | 15 ± 1 | 11.8 ± 0.1 |
| Strain 172 | 0.49 | 1.0 ± 0.5 | 8.5 ± 0.2 | 11.3 ± 0.2 |

^{a}Results expressed as mean and range of duplicate samples.

This organism served as a primary host for the attempted mshC knockout experiments. Using this host 6 of 12 or 50% of the hygromycin resistant transformants were found to be authentic knockouts in the original copy of mshC (Table 5). Strains identified as 14, 16, 18, 157, 158, and 172 all lack the original copy of mshC, as was seen in a Southern blot, but do contain the second copy of the gene. These strains also make parental levels of mycothiol and its precursor GlcN-Ins (Table 5), demonstrating the second mshC gene if fully functional. In one case, strain 3, it appears that the second copy and not the original copy of mshC was interrupted by the knockout phasmid. As a positive control for the transformation efficiency, a transformation of M. tuberculosis Erdman (without a second corA copy) with a phasmid carrying a knockout construct for the non-essential magnesium transporter corA was successful in knocking out corA in 6 of 18 or 33% of the transformants (results not shown). An empty plasmid pCV125 control strain of M. tuberculosis host Erdman was transformed with the mshC knockout phasmid (phAEΔmshC) and 0 out of 7 of the transformants were authentic mshC knockouts by Southern blot (results not shown). Thus, no transformants can be found without mycothiol in any of the above experiments. Additionally no authentic mshC knockout strains can be found by Southern analysis, except when a second copy of mshC is supplied. These results clearly support that mshC specifically, and mycothiol generally, are essential for the survival of M. tuberculosis in laboratory culture. These experiments demonstrate the essentiality of mycothiol and validate the mycothiol biosynthesis genes as possible drug targets.

TABLE 5
M. tuberculosis Erdman directed knockouts in mycothiol biosynthesis
gene mshC (Rv 2130c).

| | Knockouts by | Transformants | Knockout |
|---|---|---|---|
| | Southern blot | Examined^{a} | Efficiency |
| M. tuberculosis Erdman + | 0 | 7 | 0% |
| empty vector (for 2x mshC) | | | |
| (negative control) | | | |
| M. tuberculosis Erdman + | 6 | 18 | 33% |
| phasmid phAEΔcorA | | | |
| (positive control) | | | |
| M. tuberculosis Erdman + | 0 | 67 | 0% |
| phasmid phAEΔmshC | | | |
| M. tuberculosis Erdman | 6 | 12 | 50% |
| 2x mshC (strain 1682) + | | | |
| phasmid phAEΔmshC | | | |

^{a}Transformants are hygromycin resistant colonies appearing within 5 weeks.

### Example 4

### Screening for Inhibition of Mycothiol Production in M. smegmatis

In initial studies several fluorescent labeling agents were tested using the parental strain mc²155 of M. smegmatis that produces normal levels of MSH (˜10 μmol per g residue dry weight, defined as 100%) and mutant strain 49 that is defective in mshA and produces no MSH (<0.04% of the parental strain) or intermediates of MSH biosynthesis. Fluorescent labeling agents known to penetrate cells were examined: diethylamino-3-(4′-maleimidylphenyl)-4-methylcoumarin (CPM), monobromobimane (mBBr), and monochlorobimane (mBCl), listed in decreasing order of reactivity with thiols. Fluorescence produced with the MSH-producing parental strain mc²155 was compared with that for the MSH-deficient mutant strain 49. The fluorescence produced by CPM and mBBr was clearly greater with strain mc²155 than with strain 49, but the level from strain 49 was half that of strain mc²155 after 20 min and both levels increased steadily with time making the discrimination between MSH-producing and MSH-deficient cells difficult.

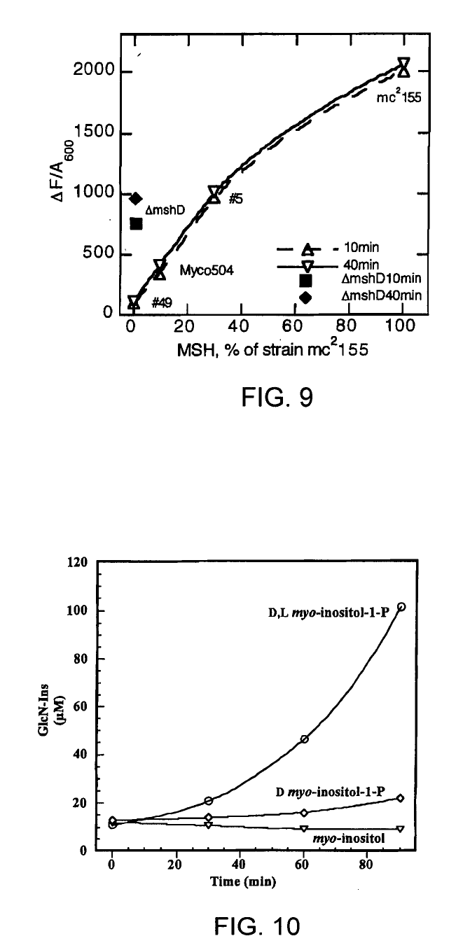

The results obtained with the substantially less reactive monochlorobimane (mBCl) were much more promising. Preliminary studies showed that addition of mBCl to 200 μl of cells at A₆₀₀=1.0 produced optimal fluorescence within minutes and acceptable subsequent upward drift in fluorescence. The concentration of mBCl required to give maximum fluorescence for the parental strain mc²155 was found to be ˜10 μM. The ratio of the fluorescence increase following addition of mBCl (ΔF) to A₆₀₀ was found to correlate with MSH content as measured by HPLC. To test whether this can be used to determine the effect of an inhibitor upon MSH content during growth of M. smegmatis, a series of mutants whose MSH contents are 0-100% of the parental strain were studied. Each strain was innoculated in medium in 96-well microplates and allowed to grow for 48 h at 37° C. The A₆₀₀ values were measured, mBCl was added to each well at 10-50 μM, and the ΔF values determined as a function of time. In this experiment a higher concentration of mBCl (35-50 μM) was required to rapidly label cellular MSH, apparently because the cells resting on the bottom of the microplate are less accessible. FIG. 8 shows the results obtained with 50 μM mBCl for five M. smegmatis strains. The relative MSH contents of these strains, as measured by HPLC analysis, are: strain mc²155, 100%; strain 5, ˜30%; strain Myco54, 5- 10%; strain ΔmshD (mshD::Tn5) 1-2%; strain 49, 0%. The results show that the fluorescence increases rapidly and then stabilizes at a value proportional to the MSH content, except for strain ΔmshD (FIG. 8). In addition to 1-2% MSH, strain ΔmshD produces formyl-Cys-GlcN-Ins at a level equivalent to 20-30% that of MSH in the parental strain. Thus, the results of FIG. 8 suggest that formyl-Cys-GlcN-Ins assays similarly to MSH but that the reaction with mBCl is slower. FIG. 9 shows that the ΔF/A₆₀₀ values remain largely constant from 10-40 min following mBCl addition and correlate well with MSH content as independently measured by HPLC. The exception is strain ΔmshD which gives a high reading owing to its formyl-Cys-GlcN-Ins content. These data show that this protocol can readily detect a 50% decrease in cellular MSH content and may be able to detect an even smaller decrease.

The rate of conversion of MSH to MSmB by mBCl in cells is orders of magnitude faster than expected from measurements of the rate of chemical reaction of mBCl with MSH (k₂=11 M⁻¹min⁻¹) at pH 7.4. It is thus apparent that a mycothiol S-transferase must be catalyzing the cellular reaction of MSH with mBCl in a similar fashion to that in which glutathione S-transferases catalyze the corresponding reaction of GSH with mBCl in mammalian cells. A possible candidate gene (Rv0274) encoding such an S-transferase has been identified. The presence of a mycothiol S-transferase facilitates the use of mBCl to screen for inhibitors of MSH biosynthesis but raises the possibility that inhibitors of the S-transferase activity would also be detected using this assay. Such mycothiol metabolism inhibitors would also be usefuf and represent a potential side-benefit of this approach.

The experiment of FIG. 8 was repeated with the parental strain mc²155 and samples taken at intervals for HPLC analysis of cells and medium for MSmB and AcCySmB content. The results showed that the cellular MSH is converted to MSmB, that MSmB is rapidly cleaved to AcCySmB, and that the AcCySmB is excreted into the medium. After 25 min of incubation >95% of the MSH in the cell was converted to AcCySmB and excreted into the medium. This is the same result as found previously with the more reactive mBBr. Following depletion of MSH with either mBBr or mBCl, M. smegmatis cells recover and grow to normal cell densities. The enzyme mycothiol S-conjugate amidase (Mca) catalyzes cleavage of MSmB to produce AcCySmB and GlcN-Ins.

### Example 5

### High Throughput Screening Assay for Inhibitors of MshA

In previous studies it was shown that GlcNAc-Ins is an intermediate in MSH biosynthesis and that MshA, a glycosyltransferase, was required for GlcNAc-Ins production. Initial attempts to identify the MshA substrates using crude extracts of M. smegmatis with uridine diphosphate N-acetyl glucosamine (UDPGlcNAc) and inositol-1-phosphate (Ins-1-P) were unsuccessful due to the incorrect stereoisomer of commercially available Ins-1-P. To identify the substrates the assay outlined below was used, which required the activity from up to three enzymes in dialyzed crude extracts of M. smegmatis and was quantitated by analysis of GlcN-Ins using amine fluorescent tagging and HPLC.

It was observed that substantial production of GlcN-Ins occurs when the commercially available 1D-Ins-1-P (from phosphatidyl inositol) is substituted with synthetic 1D,L-Ins-1-P. The stereochemistry of mycothiol requires that 1 L-Ins-1-P (also called 1D-Ins-3-P) and not 1D-Ins-1-P be the substrate for MshA. FIG. 10 shows the results for reaction of 1 mM UDP-GlcNAc with various 1 mM inositol acceptors catalyzed by a dialyzed unfractionated crude extract of M. smegmatis mc²155. Since 1D,L-Ins-1-P is a racemic mixture the 1L-Ins-1-P is present at half the total concentration or 0.5 mM. The results show that the inositol acceptor for MshA is 1 L-Ins-1-P, and that little or no activity is observed with Ins or 1D-Ins-1-P. Unlike the inositol monophosphatases (IMPases) that will utilize either 1L-Ins-1-P or 1D-Ins-1-P as substrates, MshA is stereospecific for 1L-Ins-1-P as the acceptor.

With the substrates of MshA identified a direct assay was needed for MshA activity that did not depend on the coupled enzymes required for the GlcN-Ins assay above. An HPLC assay that separates phosphates based upon the number of phosphate residues was developed and permitted the assay of the MshA catalyzed reaction based upon determination of the production of UDP from UDP-GlcNAc using A₂₆₀ detection. In this assay MshA has optimal activity with 10 mM Mg²⁺.

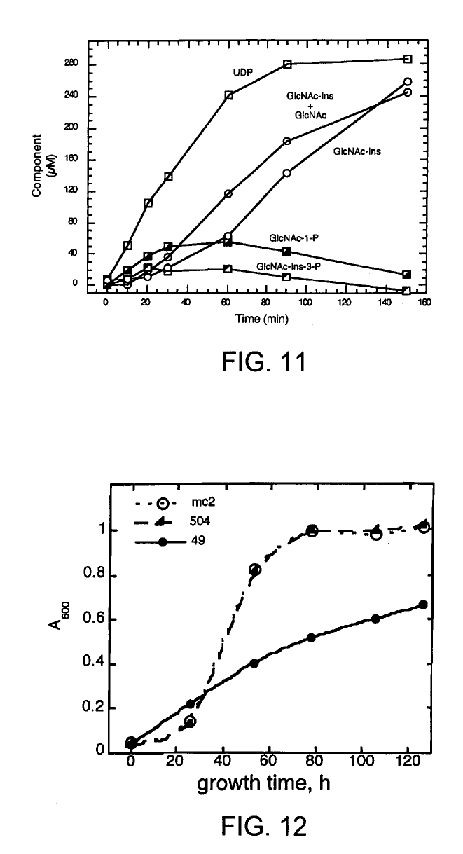

In order to identify the product of the MshA catalyzed reaction we used ³H-UDPGlcNAc [Glucosamine-6-³H(N)] to follow the glucosamine moiety in the production of GlcNAc-Ins. A dialyzed crude extract supernatant of M. smegmatis Myco504, an MshB mutant, was used to restrict the deacetylation of GlcNAc-Ins (FIG. 11). The extract was assayed with 1 mM ³H-UDPGlcNAc and 1 mM D,L Ins-1-P at times up to 150 min. Two radiolabeled monophosphate intermediates were detected that became dephosphorylated to produce GlcNAc and GlcNAc-Ins eluting unretained at ˜3 min. One intermediate was identified as GlcNAc-1-P, presumed to be generated by phosphodiesterase activity in the crude extract and to be dephosphorylated to generate GlcNAc. The other intermediate was assigned as GlcNAc-Ins-3-P, the product of the MshA catalyzed reaction, and this was verified by mass spectral analysis of this peak in an experiment using unlabeled UDP-GlcNAc. A combination of radioactive HPLC monitoring, UDP-GlcNAc and UDP (A₂₆₀) analysis, and specific GlcNAc-Ins analysis was used to generate the reaction profile and to produce the data shown in FIG. 11. Over the 150 min incubation ˜350 μM UDPGlcNAc was consumed and produced ˜290 μM UDP and 250 μM GlcNAc-Ins for an overall ˜50% conversion of 1L-Ins-1-P to product. The counts eluting at 3 min measure unretained GlcNAc-Ins and a small amount of GlcNAc generated by dephosphorylation of GlcNAc-1-P. These results establish that GlcNAc-Ins-3-P is the product of the MshA catalyzed reaction and that a previously unidentified phosphatase required for MSH biosynthesis, designated MshA2, is required to generate GlcNAc-Ins.

When MshA2 is identified and both MshA and MshA2 are cloned and expressed, then a simple coupled enzyme screening assay can be devised. For each equivalent of 1L-Ins-1-P and UDPGlcNAc consumed there will be one equivalent of Pᵢ produced that can be quantified with the malachite green colorimetric assay developed for MshC. The required substrates for the MshA+MshA2 screen are commercially available. It is possible that MshA2 is also essential for MSH biosynthesis and for growth of M. tuberculosis but this has not yet been demonstrated. If so, then the assay would potentially detect inhibitors of two enzymes essential for MSH biosynthesis. A counterscreen to detect inhibitors of MshA2 is needed and will be developed. As a secondary screen to confirm that MshA is the target the MshA specific HPLC assay of UDP production can be used as described above.

### Example 6

### Screening for Inhibition of Mycothiol Production in M. smegmatis Based on Resistance to Isoniazid (INH)

It was observed during the initial studies provided herein relating to mutants of M. sinegmatis that mutant strain 49, which does not produce MSH, was highly resistant to INH. Based on this property a Tn5 transposon mutant library was screened to select for MSH mutant clones deficient in MSH, paving the way for identification of the mshD and mshA genes of MSH biosynthesis. Accordingly, the present study establishes the basis for using INH resistance to test for inhibition of MSH biosynthesis in M. smegmatis.

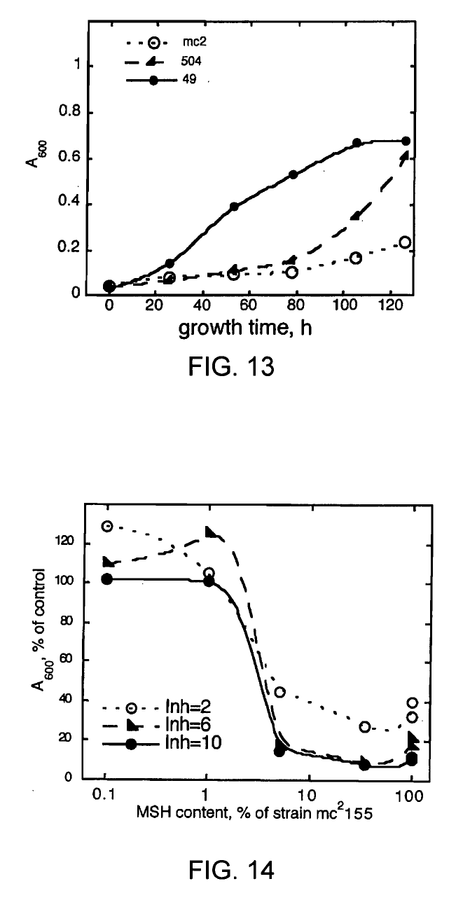

Using the parental strain mc²155, the Myco504 mutant in mshB that produces 5-10% of normal MSH in exponential growth, and the mutant strain 49 that produces 0% of normal MSH, the effect of 0-30 μg per ml INH upon growth was examined. In the absence of INH (FIG. 12) strains mc²155 and Myco504 exhibit identical growth whereas strain 49 grows more slowly. At 2 μg per ml INH and above growth of strain mc²155 became inhibited but strain 49 shows no effect. This is seen in FIG. 13 for growth in the presence of 10 μg per ml INH. Growth of strain mc²155 is markedly inhibited whereas strain 49 grows at the same rate as in the absence of INH. Growth of strain Myco504 is also markedly inhibited but growth surges after 80 h.

Based upon the results of FIGS. 12 and 13˜10 μg per ml INH and ˜80 h were selected as the optimal conditions for testing MSH production based upon resistance to INH. Using additional mutant strains of M. smegmatis blocked in MSH production, strain I64(1% MSH) and strain 5 (˜30% MSH), the relationship between MSH content and growth in the presence of 2-12 μg per ml INH was examined (FIG. 14). The results show that growth becomes sharply attenuated at MSH concentrations between 1% and 5% of the normal value. INH concentrations of 6 and 10 μg per ml gave similar results, as did 12 μg per ml INH. As such, this provides the basis for using growth in the presence of INH and a potential inhibitor of MSH biosynthesis as the basis for screening for strong inhibitors of MSH biosynthesis. It is more limited than the mBCl assay and may therefore be developed for use as a secondary screen, or as an alternative primary screen.

The biochemical basis of the requirement for MSH to produce sensitivity isoniazid is not understood. It may involve an enzymatic step dependent upon MSH. If so, then this enzyme might be inhibited by one of the compounds in a library being screened with this technique and give a false positive result in this assay. Such a false positive, or those arising from other identified sources of INH resistance, would be detected in the secondary screening assays available to identify the specific target of inhibition in the MSH biosynthetic pathway.

### Example 7

### Secondary Screening by HPLC Assay

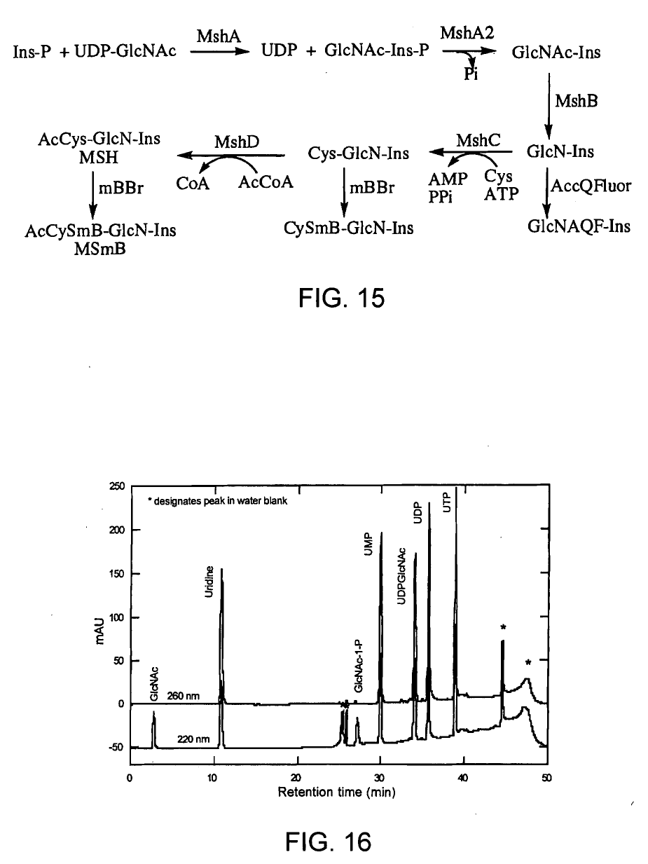

Mycothiol is synthesized in five sequential enzymatic steps (FIG. 15). In the course of over a decade of study of mycothiol biochemistry the inventors have developed assays for the various intermediates in the MSH biosynthesis pathway and these assays can be used to identify the target of an inhibitor based upon accumulation of the biosynthetic intermediate.

Fluorescent labeling and HPLC analysis with fluorescence detection allows sensitive determination of GlcN-Ins, Cys-GlcN-Ins, and MSH. Determination of GlcN-Ins before and after treatment of the extract with MshB to convert GlcNAc-Ins to GlcN-Ins allows the assessment of GlcNAc-Ins by difference.

Application of these methods to the M. smegmatis mutants blocked at various steps in the biosynthesis pathway illustrates the results expected for an inhibitor that interferes with a specific enzyme (Table 6). In the parent strain the MSH and GlcN-Ins levels are substantial while the GlcNAc-Ins and Cys-GlcN-Ins levels are below readily measured levels. In the mshA::Tn5 mutant (ΔmshA) no product or intermediates are formed. The Myco504 (ΔmshB) mutant produces low MSH levels but has a markedly elevated GlcNAc-Ins level and a substantially depleted GlcN-Ins level. Various mutants in MshC (ΔmshC) exhibit minimal MSH production but show elevated GlcN-Ins levels. Finally, in the transposon mutant mshD::Tn5 (ΔmshD), having no MshD activity, the Cys-GlcN-Ins level jumps ˜100-fold while the MSH level drops 200-fold. From Table 6 it is clear that determination of the levels of MSH and its immediate precursors for cells grown in the presence of an inhibitor that interferes with MSH biosynthesis will serve to identify which of these enzymes is inhibited. Such analyses will validate the cell screening results for inhibition of MSH biosynthesis and identify the target involved.

TABLE 6
GlcNAc-Ins, GlcN-Ins, Cys-GlcN-Ins, and MSH content
(μmol per g residual dry weight) for M. smegmatis mutant strains

| Strain | GlcNAc-Ins | GlcN-Ins | Cys-GlcN-Ins | Msh |
|---|---|---|---|---|
| ΔmshA | <0.01 | <0.01 | <0.01 | <0.01 |
| ΔmshB | 2.6 | <0.001 | <0.01 | 0.4-1 |
| ΔmshC | — | 2-3 | <0.01 | <0.1 |
| ΔmshD | — | — | 0.5-2.0 | ~0.5 |
| Parent mc²155 | <0.2 | 0.1-1 | <0.01 | 9-10 |

### Example 8

### Inhibition of the Growth of M. tuberculosis

This example illustrates that compound NTF 1836 inhibits the growth of M. tuberculosis.

Bacteria in broth cultures were incubated with dilutions of compound NTF 1836 for 7 days. Increase in bacterial numbers was assessed using two methods: by measuring the optical density of the cultures and by plating dilutions of the cultures on Middlebrook agar plates. The OD₆₀₀ of the starting culture was 0.08. The IC₅₀ of NTF 1836 for M. tuberculosis was between 20 uM and 30 uM as measured by both optical density and by plate counts of viable bacteria (Table 7).

Compound NTF 1836 inhibits the activity of MshC within M. tuberculosis. Evidence for the inhibition of MshC comes from the decrease in mycothiol and the increase in GlcN-Ins, the substrate for MshC, relative to mycothiol concentration. This is most readily seen by the increase in ratio of GlcN-Ins to mycothiol at 50 uM and 75 uM NTF 1836.

TABLE 7
Day 7 samples from M. tuberculosis incubated
with NTF1836 (triplicates)

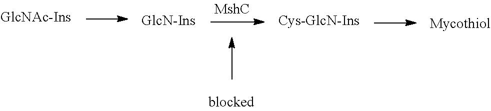

| | | mycothiol | GlcN-Ins | |
|---|---|---|---|---|
| Conc. | | (nmoles/10⁹ | (nmoles/10⁹ | GlcN-Ins/ |
| NTF 1836 | OD₆₀₀ | bacteria | bacteria | mycothiol |
| 0 | 1.654 ± 0.046 | 20.2 ± 1.2 | 6.41 ± 0.62 | 0.317 |
| 20 uM | 1.304 ± 0.014 | 27.8 ± 2.5 | 2.77 ± 0.09 | 0.09 |
| 30 uM | 0.600 ± 0.049 | 19.1 ± 2.5 | 3.49 ± 0.10 | 0.18 |
| 50 uM | 0.271 | 10.0 | 4.28 | 0.428 |
| 75 uM | 0.12 | 3.5 | 4.22 | 1.21 |

In addition to MshC, it is believed that NTF1836 inhibits a related enzyme that serves as a second target for killing of M. tuberculosis. This second target is most likely cysteine t-RNA synthetase, a protein with a closely related structure to MshC. The evidence for this comes from the fact that the IC₅₀ of NTF 1836 for MshC activity in an vitro enzyme assay is 100 uM while the IC₅₀ for growth inhibition of M. tuberculosis is between 20 uM and 30 uM. The first half reaction for MshC and Cys tRNA synthase is identical and involves the formation of an enzyme bound cysteinyl-adenylate. A well known nM transition state analog inhibitor of Cys tRNA synthase (C. Evilia and Y. M. Hou, (2006) Biochemistry 45, 6835-6845), 5′-O-[N-(L-cysteinyl)sulfamoyl]adenosine, also inhibits MshC with an IC₅₀ of ˜40 nM when assayed under the conditions described in [0186]. Thus, it is likely that inhibitors of MshC will be inhibitors of Cys tRNA synthase. Additional evidence for Cys tRNA synthase as a second target for NTF1836 and homologs is shown in Table 8 for growth inhibition of Gram-positive pathogens. NTF 1836 was found to be inhibitory for an actinomycete where mycothiol biosynthesis is non-essential (M. smegmatis) and for Gram-positive pathogens such as Staphylococcus and Enterococcus where mycothiol biosynthesis is missing (Staphylococcus aureus and Enterococcus faecalis). The data in Table 8 indicate that NTF1836 and homologs have broad range Gram-postive antibacterial activity, which is consistent with the inhibition of Cys tRNA synthase, a validated antibacterial drug target (D. J. Payne et al, (2007) Nature Rev. Drug Discovery 6, 29-40).

TABLE 8
Drug suseptiblity of gram positive pathogens to NTF1836,
MIC by broth dilution assay (n = 3)

| | | MIC |
|---|---|---|
| Organism | Medium | (μg/ml) |
| Mycobacterium smegmatis mc²155 | Middlebrook 7H9¹ | 16 |
| ATCC 700084 | | |
| Staphylococcus aureus RN450 | TSB² | 64 |
| Staphylococcus aureus ATCC 25923 | TSB² | 128 |
| Enterococcus faecalis ATCC 29212 | Todd Hewitt³ | 64 |

¹Middlebrook 7H9 + 1% glucose
²TSB = trypticase Soy Broth, BBL
³Todd Hewitt-Difco

### Example 9

### Inhibitor Homologs of NTF1836

A screen of ˜500 homologs of NTF1836 available from Chemical Diversity that have varying X, Y, and Z functionalities. All compounds were also counterscreened for phosphatase inhibition and the positive hits in the MshC screen were tested in the HPLC assay. This allows for an evaluation of the adequacy of the pyrophosphatase counterscreen to reduce false positives. NTF1836 is identified by the following formula:

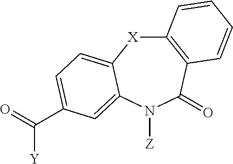

where X═SO and Y=

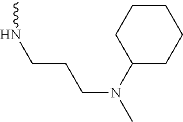

### and Z=

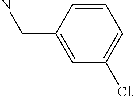

Compounds were tested for inhibition of MshC in 25 mM histidine buffer pH 8.0 containing 50 mM NaCl and 5 mM MgSO₄ at 23° C. Substrate concentrations were 100 μM for Cys (Kₘ˜85 μM), 50 μM for GlcN-Ins (Kₘ˜280 μM), and 100 μM for ATP (saturating). Initial rates were determined by labeling of thiols with monobromobimane and analysis of thiol content by HPLC with fluorescence detection (G. L. Newton, P. Ta, D. Sareen, and R. C. Fahey, Anal. Biochem. 353:167-173 (2006)). Initial rates (<30% conversion of GlcN-Ins) were calculated from Cys-GlcN-Ins production and IC₅₀ values were estimated from a plot of 1/rate vs. inhibitor concentration. This assay directly measures the MshC catalyzed reaction and is not subject to the artifactual inhibition that can occur in coupled enzyme assays.

The results of screening NTF1836 homologs are presented in Tables 9 and 10. Significant inhibition is achieved with X═S (#1754, 1776, 1779, 1806 and 1937 in Table 9), X═SO (#1028, 1051, 1061, 1137, 1938 in Table 9; #83-07, 08, 11, 60, 64, 65, 66, 70, 72, 73, 75 in Table 10), and X═SO₂ (1139, 1947, 1954, 1965 in Table 9; 83-27, 28, 35, 39, 40, 54, 78, 83 in Table 10). Measurable inhibition is achieved when Z is a benzyl moiety bearing one or more halogens at various sites in the benzene ring (#1028, 1051, 1137, 1139, 1776, 1779, 1806, 1937, 1938, 1947, 1954, 1965 in Table 9; #83-27, 28, 39, 40, 58, 64, 65. 66, 70, 72, 73, 75, 78, 83 in Table 10). Weak but detectable inhibition is detected when the Z benzyl moiety contains methyl groups in the 2,5 positions (#1061, 1062 in Table 9; 83-07 in Table 10) and in one example (83-54, Table 10) when the benzyl moiety is unsubstituted. With suitable X and Z groups, compounds having a 1,3-diaminopropyl residue in the Z-moiety forming an amide bond to the ring exhibit measurable inhibition if the amino residue remote from the ring is capable of protonation at physiologic pH (#1028, 1051, 1061, 1062, 1137, 1139, 1776, 1779, 1937, 1938, 1954, 1965 in Table 9; 83-07, 11, 27, 28, 39, 40, 44, 54, 64, 65, 66, 70, 72, 73, 75, 78, 83 in Table 10). Similar compounds lacking the basic amino group produce little or no inhibition (#1920, 1939, 1964 in Table 9; 83-12, 47, 55, 61, 74 in Table 10) as do compounds bearing groups containing in excess of 10 carbons attached to the remote amino residue (#1940, 1949 in Table 9; 83-02, 26, 32, 33, 42, 52, 56, 62, 69, 76, 79 in Table 10).

TABLE 9
Homologs of NTF1836

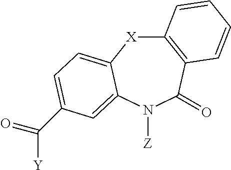

| Inhibitor # | IC₅₀, μM | X | Y | Z |
|---|---|---|---|---|
| 1028 | 335 ± 15 | SO | 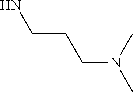 | 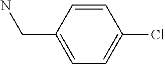 |
| 1051 | 710 ± 20 | SO | 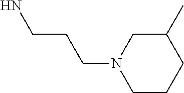 | 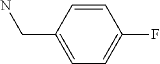 |
| 1061 | 1600 ± 300 | SO | 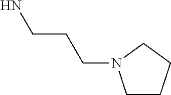 | 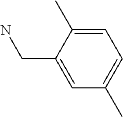 |
| 1062 | ~2500 | SO | 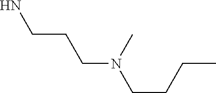 | 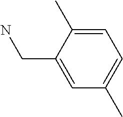 |
| 1075 | >3000 | SO | 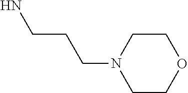 | 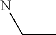 |
| 1081 | >3000 | SO | 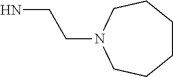 | 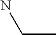 |
| 1137 | 1400 ± 700 | SO | 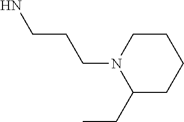 | 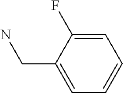 |
| 1138 | >3000 | SO₂ | 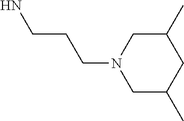 | 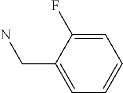 |
| 1139 | 340 ± 60 | SO₂ | 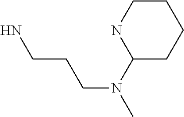 | 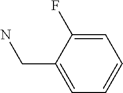 |
| 1173 | >2000 | SO₂ | 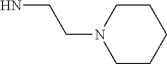 | 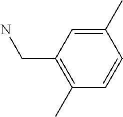 |
| 1458 | ~3000 | S | 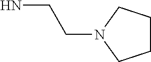 | 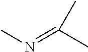 |
| 1671 | >3000 | S | 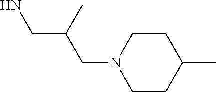 | 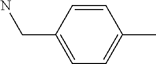 |
| 1672 | >3000 | S | 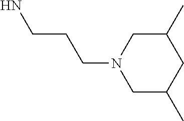 | 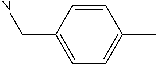 |
| 1751 | >3000 | S | 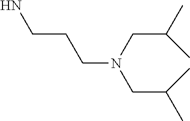 | 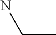 |
| 1754 | ~2000 | S | 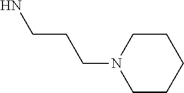 | 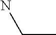 |
| 1776 | 320 ± 50 | S | 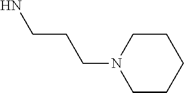 | 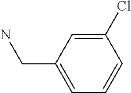 |
| 1779 | 135 ± 30 | S | 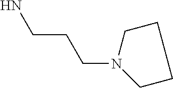 | 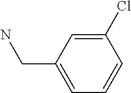 |
| 1781 | >3000 | S | 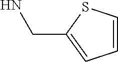 | 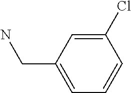 |
| 1806 | ~750 | S | 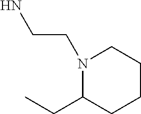 | 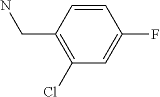 |
| 1841 | >3000 | S | 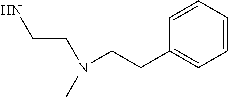 | 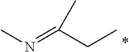 |
| 1864 | >3000 | S | 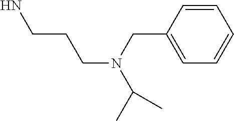 | 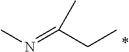 |
| 1866 | >3000 | S | 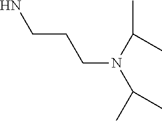 | 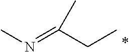 |
| 1920 | >3000 | S | 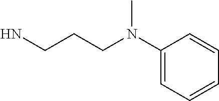 | 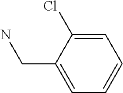 |
| 1937 | 780 ± 120 | S | 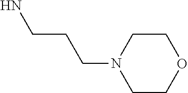 | 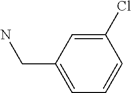 |
| 1938 | 102 ± 4 | SO | 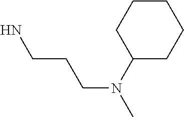 | 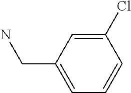 |
| 1939 | 3200 ± 1000 | SO | 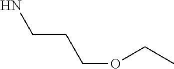 | 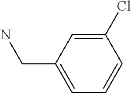 |
| 1940 | >3000 | SO | 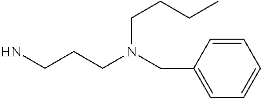 | 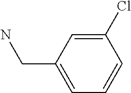 |
| 1943 | >3000 | SO₂ | 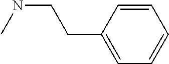 | 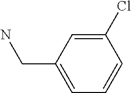 |
| 1947 | 2300 ± 300 | SO₂ | 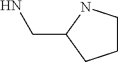 | 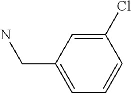 |
| 1949 | >3000 | SO₂ | 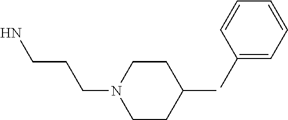 | 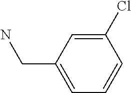 |
| 1954 | 1100 ± 200 | SO₂ | 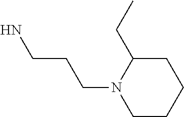 | 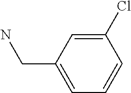 |
| 1964 | >3000 | SO₂ | 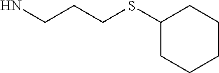 | 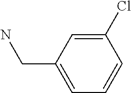 |
| 1965 | 1700 ± 300 | SO₂ | 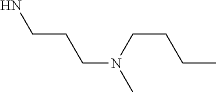 | 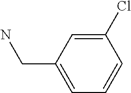 |
| 1966 | >3000 | SO₂ | 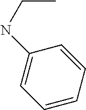 | 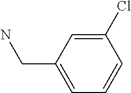 |
| 1984 | >3000 | SO | 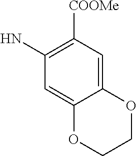 | 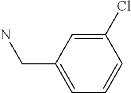 |
| 1986 | >3000 | SO | 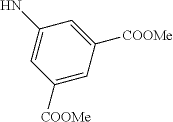 | 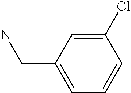 |

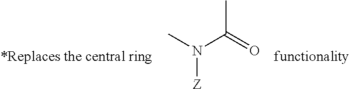

TABLE 10
Additional Homologs of NTF1836

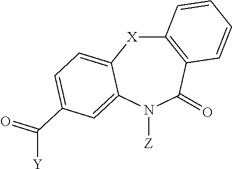

| Inhibitor # | IC₅₀ | | X | Y | Z |
|---|---|---|---|---|---|
| 83-01 | >3 | mM | SO | 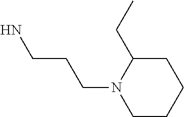 | 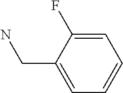 |
| 83-02 | >3 | mM | SO | 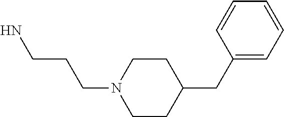 | 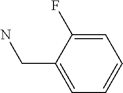 |
| 83-03 | >3 | mM | SO | 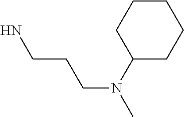 | 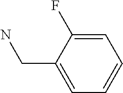 |
| 83-04 | >3 | mM | SO | 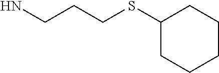 | 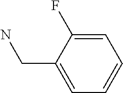 |
| 83-05 | >3 | mM | SO | 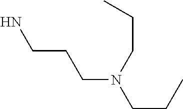 | 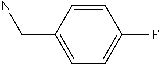 |
| 83-06 | >3 | mM | SO | 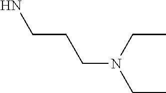 | 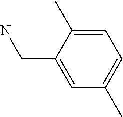 |
| 83-07 | 650 ± 50 | μM | SO | 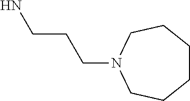 | 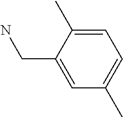 |
| 83-08 | ~3 | mM | SO | 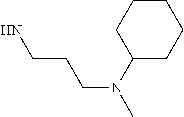 | 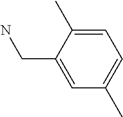 |
| 83-09 | >3 | mM | SO | 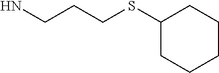 | 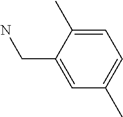 |
| 83-10 | >3 | mM | SO | 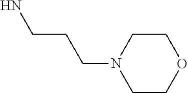 | 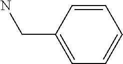 |
| 83-11 | 400 ± 100 | μM | SO | 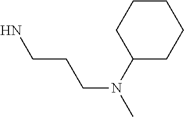 | 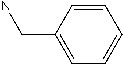 |
| 83-12 | >3 | mM | SO | 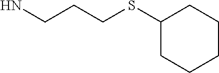 | 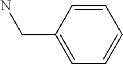 |
| 83-13 | >3 | mM | SO | 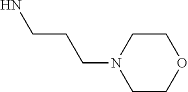 | 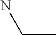 |
| 83-14 | >3 | mM | SO | 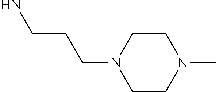 | 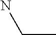 |
| 83-15 | >3 | mM | SO | 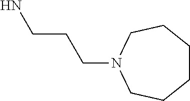 | 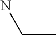 |
| 83-16 | >3 | mM | SO | 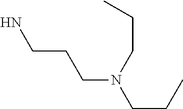 | 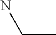 |
| 83-17 | >3 | mM | SO | 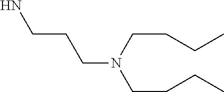 | 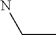 |
| 83-18 | >3 | mM | SO | 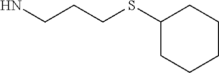 | 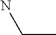 |
| 83-19 | ~3 | mM | SO₂ | 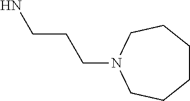 | 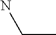 |
| 83-20 | >3 | mM | SO₂ | 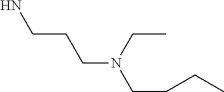 | 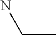 |
| 83-21 | ~2 | mM | SO₂ | 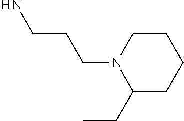 | 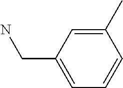 |
| 83-22 | >3 | mM | SO₂ | 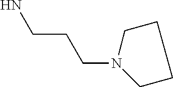 | 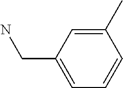 |
| 83-23 | >3 | mM | So₂ | 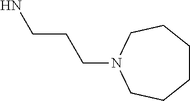 | 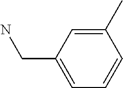 |
| 83-24 | 1.5 ± 0.5 | mM | SO₂ | 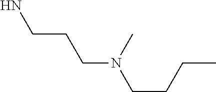 | 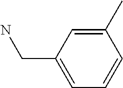 |
| 83-25 | >3 | mM | SO₂ | 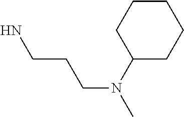 | 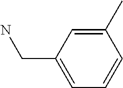 |
| 83-26 | >3 | mM | SO₂ | 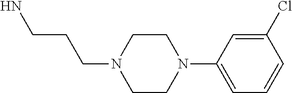 | 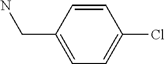 |
| 83-27 | 700 ± 250 | μM | SO₂ | 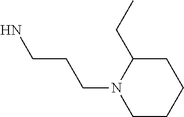 | 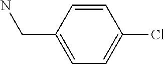 |
| 83-28 | 250 ± 100 | μM | SO₂ | 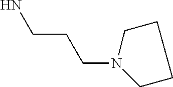 | 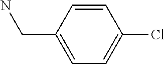 |
| 83-29 | >3 | mM | SO₂ | 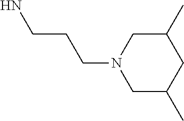 | 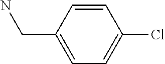 |
| 83-30 | >3 | mM | SO₂ | 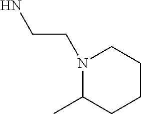 | 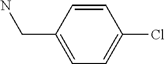 |
| 83-31 | 3 | mM | SO₂ | 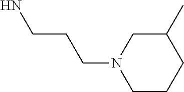 | 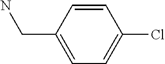 |
| 83-32 | ~2 | mM | SO₂ | 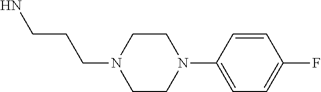 | 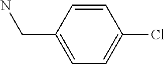 |
| 83-33 | >3 | mM | SO₂ | 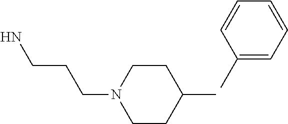 | 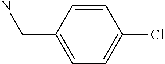 |
| 83-34 | >3 | mM | SO₂ | 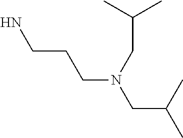 | 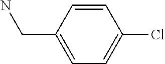 |
| 83-35 | 350 ± 40 | μM | SO₂ | 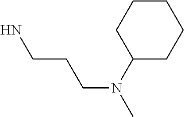 | 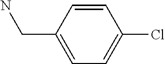 |
| 83-36 | >3 | mM | SO₂ | 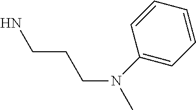 | 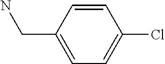 |
| 83-37 | >500 | mM | SO₂ | 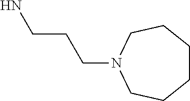 | 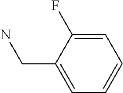 |
| 83-38 | >500 | mM | SO₂ | 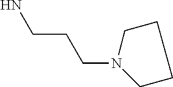 | 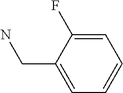 |
| 83-39 | 100 ± 20 | μM | SO₂ | 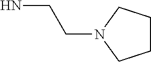 | 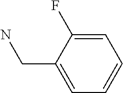 |
| 83-40 | 100 ± 30 | μM | SO₂ | 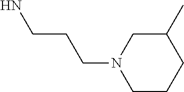 | 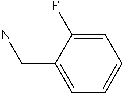 |
| 83-42 | >3 | mM | SO₂ | 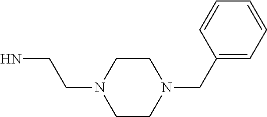 | 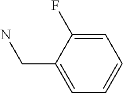 |
| 83-43 | >3 | mM | SO₂ | 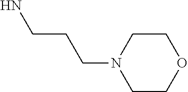 | 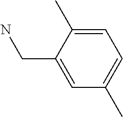 |
| 83-44 | >500 | μM | SO2 | 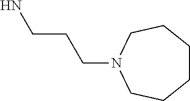 | 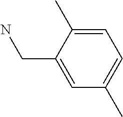 |
| 83-45 | >3 | mM | SO₂ | 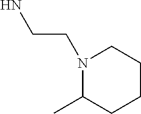 | 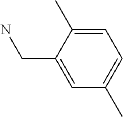 |
| 83-46 | ≧500 | μM | SO2 | 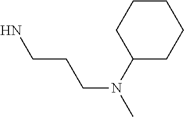 | 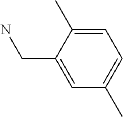 |
| 83-47 | >3 | mM | SO2 | 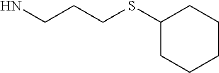 | 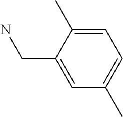 |
| 83-48 | ≧500 | μM | SO₂ | 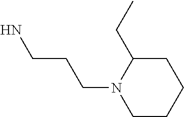 | 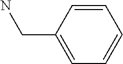 |
| 83-49 | ~2 | mM | SO₂ | 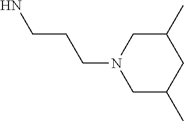 | 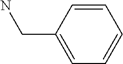 |
| 83-50 | >3 | mM | SO₂ | 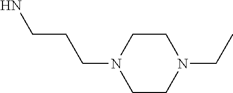 | 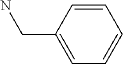 |
| 83-51 | 1.7 ± 0.4 | mM | SO₂ | 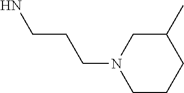 | 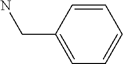 |
| 83-52 | 2.5 ± 1.2 | mM | SO₂ | 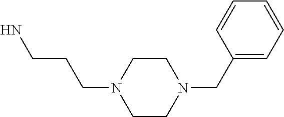 | 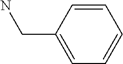 |
| 83-53 | >3 | mM | SO₂ | 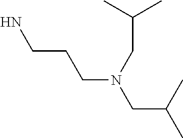 | 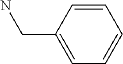 |
| 83-54 | 510 ± 80 | μM | SO₂ | 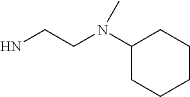 | 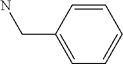 |
| 83-55 | >3 | mM | SO₂ | 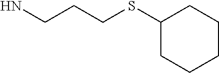 | 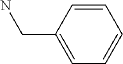 |
| 83-56 | >3 | mM | SO | 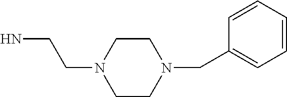 | 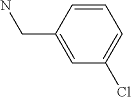 |
| 83-57 | ~1 | mM | SO₂ | 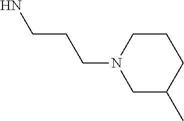 | 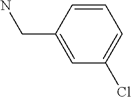 |
| 83-58 | ≧500 | μM | SO₂ | 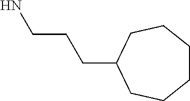 | 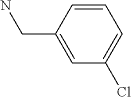 |
| 83-59 | >3 | mM | SO₂ | 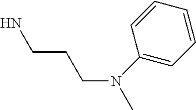 | 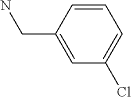 |
| 83-60 | ÷500 | μM | SO | 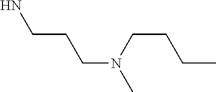 | 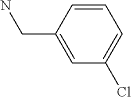 |
| 83-61 | ~1 | mM | SO | 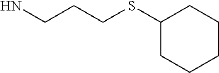 | 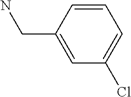 |
| 83-62 | 2.1 ± 0.9 | mM | SO | 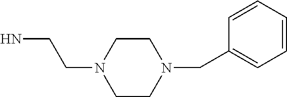 | 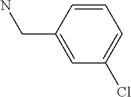 |
| 83-63 | >2 | mM | SO | 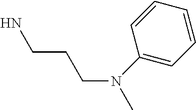 | 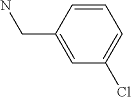 |
| 83-64 | 150 ± 30 | μM | SO | 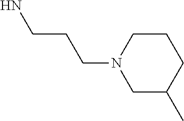 | 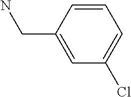 |
| 83-65 | 500 ± 60 | μM | SO | 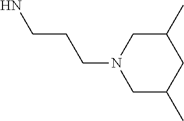 | 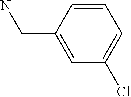 |
| 83-66 | 80 ± 20 | μM | SO | 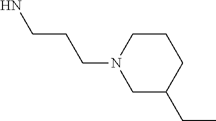 | 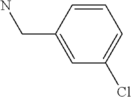 |
| 83-67 | ~3 | mM | SO | 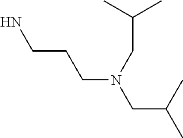 | 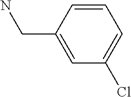 |
| 83-68 | 1.5 ± 0.5 | mM | SO | 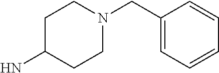 | 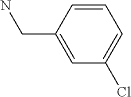 |
| 83-69 | 2.5 ± 0.7 | mM | SO | 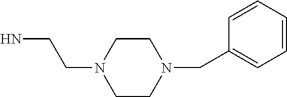 | 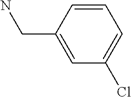 |
| 83-70 | 470 ± 130 | μM | SO | 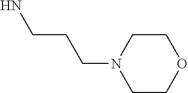 | 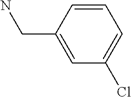 |
| 83-72 | 97 ± 26 | μM | SO | 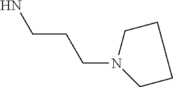 | 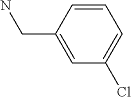 |
| 83-73 | ~200 | μM | SO | 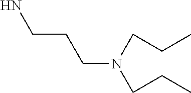 | 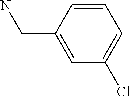 |
| 83-74 | >3 | mM | SO | 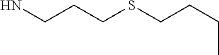 | 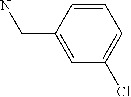 |
| 83-75 | ~80 | μM | SO | 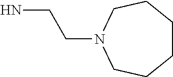 | 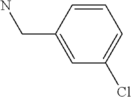 |
| 83-76 | ≧3 | mM | SO | 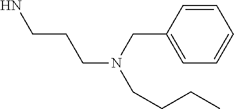 | 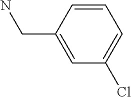 |
| 83-77 | 2.3 ± 0.7 | mM | SO₂ | 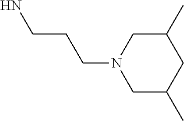 | 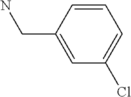 |
| 83-78 | ~500 | μM | SO₂ | 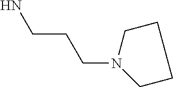 | 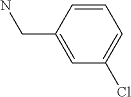 |
| 83-79 | >3 | mM | SO₂ | 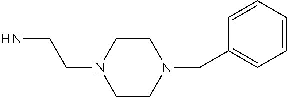 | 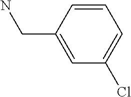 |
| 83-80 | >3 | mM | SO₂ | 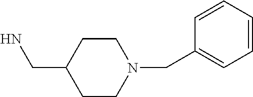 | 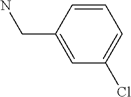 |
| 83-81 | >3 | mM | SO₂ | 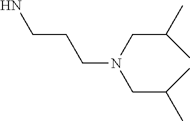 | 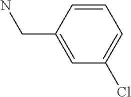 |
| 83-82 | ~3 | mM | SO₂ | 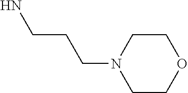 | 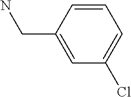 |
| 83-83 | 980 ± 40 | μM | SO₂ | 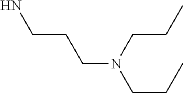 | 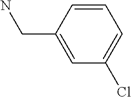 |

Although the invention has been described with reference to the above examples, it will be understood that modifications and variations are encompassed within the spirit and scope of the invention. Accordingly, the invention is limited only by the following claims.

What is claimed is:

1. A compound of the formula: 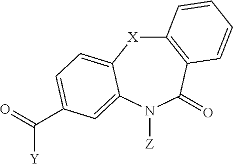
where X═SO, Y= 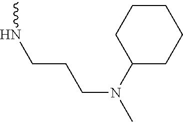
and Z=

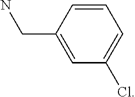

2. A derivative or homolog of the compound of claim 1, wherein the derivative or homolog is listed in Table 9 or Table 10.

3. A compound that effectively inhibits mycothiol biosynthesis enzymes MshA, MshC or MshD or Cys tRNA synthase activity in a microorganism.

4. The compound of claim 3, wherein the microorganism is a Gram-positive microorganism.

5. The compound of claim 3, wherein the compound is NTF1836.

6. The compound of claim 3, wherein the compound is listed in Table 9 or Table 10.

7. A pharmaceitucal composition comprising an inhibitor of mycothiol biosynthesis enzymes MshA, MshC or MshD or Cys tRNA synthase activity of a microorganism.

8. The pharmaceutical composition of claim 7, wherein the inhibitor is NTF 1836.

9. The pharmaceutical composition of claim 7, wherein the inhibitor is any inhibitor listed in Table 9 or Table 10.

10. A method for identifying an inhibitor of mycothiol biosynthesis comprising:
a) contacting a candidate compound for inhibition of MshC, MshD or MshA with a mycothiol-producing bacterium, under suitable conditions, and
b) determining the presence or absence of mycothiol within the mycothiol- producing bacterium,
wherein a substantial absence of mycothiol is indicative of a candidate compound that inhibits MshC, MshD or MshA and thereby inhibits mycothiol biosynthesis.

11. The method of claim 10, wherein the bacterium is an actinomycete.

12. The method of claim 10, wherein the candidate compound is a polypeptide, polynucleotide or small molecule.

13. The method of claim 10, wherein the inhibition of mycothiol biosynthesis is by inhibition of Msh cysteine:glucosaminyl ligase.

14. The method of claim 10, wherein the inhibition of mycothiol biosynthesis is by inhibition of MshD acetyl-CoA:Cys-GlcN-Ins acetyltransferase.

15. The method of claim 10, wherein the inhibition of mycothiol biosynthesis is by inhibition of MshA glycosyltransferase.

16. An inhibitor of mycothiol biosynthesis identified by the method of claim 10.

17. A method for inhibiting mycothiol biosynthesis comprising contacting, under suitable conditions, a mycothiol-producing bacterium with NTF1836 or a homolog thereof.

18. The method of claim 17, wherein the NTF1836 homolog is any homolog listed in Table 9 or Table 10.

19. The method of claim 17, further comprising determining the presence or absence of mycothiol within the mycothiol-producing bacterium, wherein a substantial absence of mycothiol confirms inhibition of MshC, MshD or MshA, thereby inhibiting mycothiol biosynthesis.

20. A method for increasing sensitivity of a pathogenic mycothiol-producing bacterium in mammalian cells to an antibiotic, said method comprising:
introducing into the bacterium an inhibitor of endogenous bacterial mycothiol biosynthesis enzyme, wherein the enzyme is selected from MshC, MshD and MshA,
wherein the intracellular presence of the inhibitor decreases mycothiol biosynthesis by the bacterium in said mammalian cells as compared with untreated control bacterium so as to increase sensitivity of the bacterium to an antibiotic.

21. The method of claim 20, wherein the inhibitor inhibits MshC cysteine:glucosaminyl inositol ligase activity.

22. The method of claim 20, wherein the inhibitor inhibits MshD acetyl-CoA:Cys-GlcN-Ins acetyltransferase activity.

23. The method of claim 20, wherein the inhibitor inhibits MshA glycosyltransferase activity.

24. The method of claim 20, wherein the introducing comprises culturing the bacterium in the presence of the inhibitor.

25. The method of claim 20, wherein the inhibitor is NTF1836.

26. The method of claim 20, wherein the inhibitor is any inhibitor listed in Table 9 or Table 10.

27. A method for inhibiting growth of a mycothiol-producing bacterium in a mammal, said method comprising administering to the mammal an effective amount of an inhibitor of intracellular MshC, thereby inhibiting growth of the bacterium in the mammal.

28. The method of claim 27, wherein the bacterium is an actinomycete.

29. The method of claim 27, wherein the inhibitor is NTF1836.

30. The method of claim 27, wherein the inhibitor is any inhibitor listed in Tables 9 or 10.

31. A method of treating a Gram-positive bacterial infection in a subject in need thereof comprising administering an therapeutically effective amount of an inhibitor of intracellular MshC.

32. The method of claim 31, wherein the inhibitor is NTF1836.

33. The method of claim 31, wherein the inhibitor is any inhibitor listed in Tables 9 or 10.

34. The method of claim 31, wherein the infection is a Staphylococcus aureus infection.

35. The method of claim 31, wherein the infection is a Staphylococcus spp. infection.

36. The method of claim 31, wherein the infection is a Enterococcus faecalis infection.

37. The method of claim 31, wherein the infection is a Enterococcus spp. infection.

38. The method of claim 31, wherein the infection is a Streptococcus spp. infection.

39. A method of treating an infection due to a microorganism in a subject in need thereof comprising administering a therapeutically effective amount of a compound of claims 1 or 2 or a pharmaceutical composition of any one of claims 3-5.

40. The method of claim 39, wherein the infection is a Staphylococcus aureus infection.

41. The method of claim 39, wherein the infection is a Staphylococcus spp. infection.

42. The method of claim 39, wherein the infection is a Enterococcus faecalis infection.

43. The method of claim 39, wherein the infection is a Enterococcus spp. infection.

44. The method of claim 39, wherein the infection is a Streptococcus spp. infection.
